(19) United States
(12) Patent Application Publication
(10) Pub. No.: US 2020/0056149 A1
(43) Pub. Date: Feb. 20, 2020

(54) READY-TO-USE CRYOPRESERVED CELLS

(71) Applicant: MEMORIAL SLOAN-KETTERING CANCER CENTER, New York, NY (US)

(72) Inventors: Mark Tomishima, Chatham, NJ (US); Karen Wong, New York, NY (US)

(73) Assignee: MEMORIAL SLOAN-KETTERING CANCER CENTER, New York, NY (US)

(21) Appl. No.: 16/664,182

(22) Filed: Oct. 25, 2019

Related U.S. Application Data
(63) Continuation of application No. PCT/US2018/029529, filed on Apr. 26, 2018.
(60) Provisional application No. 62/518,891, filed on Jun. 13, 2017.
(60) Provisional application No. 62/519,006, filed on Jun. 13, 2017.
(60) Provisional application No. 62/490,432, filed on Apr. 26, 2017.

(51) Int. Cl. C12N 5/0735; A01N 1/02

(52) U.S. Cl. CPC C12N 5/0606; A01N 1/0284; C12N 2523/00; C12N 2510/00; A01N 1/0221

(57) ABSTRACT

The presently disclosed subject matter relates to compositions of ready-to-use cryopreserved populations of dissociated cells which can be directly used for downstream application without after-thaw expansion and/or passage. The presently disclosed subject matter also provides for methods of preparing such compositions, and in vitro methods of using such compositions.

### CROSS-REFERENCE TO RELATED APPLICATION

This application is a Continuation of International Patent Application No. PCT/US18/29529 filed Apr. 26, 2018, which claims priority to U.S. Provisional Application No. 62/490,432 filed on Apr. 26, 2017, U.S. Provisional Application No. 62/518,891 filed on Jun. 13, 2017, and U.S. Provisional Application No. 62/519,006 filed on Jun. 13, 2017, the contents of each of which are incorporated by reference in their entirety, and to each of which priority is claimed.

### 1. INTRODUCTION

The presently disclosed subject matter relates to compositions of ready-to-use cryopreserved cells which can be directly used for downstream applications without after-thaw expansion and/or passage, and to methods of preparing said compositions, precursor compositions, and methods of in vitro culturing of cells in said compositions.

### 2. BACKGROUND OF THE INVENTION

Human pluripotent stem cells (hPSCs) are revolutionizing disease modeling and cell replacement therapies. PSCs are now routinely made from patients (e.g., as induced PSCs, or iPSCs), and genetic alterations suspected to cause disease can be changed at will to explore genotype-phenotype relationships. These technologies are bridging genome-wide associations to causation and provide an unprecedented view into disease mechanisms when combined with the increasingly sophisticated ability to direct PSC differentiation, a necessary step to provide a disease-relevant context. Aside from disease modeling, PSCs can be directed to clinically relevant cell types to provide an unlimited resource for cell replacement therapies. While both PSC applications are powerful, there are many practical aspects of hPSC maintenance and differentiation that could be improved.

One practical complication in disease modeling experiments is maintaining hPSC lines while simultaneously directing their differentiation. In iPSC disease modeling studies, “best practices” require repeatedly expanding and differentiating many iPSC lines in parallel, a process defined here as “continuous passage”. Synchronizing PSCs is challenging since different lines often expand at different rates. Another complication is that each PSC cell line can drift during continuous passage, so poor differentiation might reflect either the disease state or suboptimal PSC culture. Continuous passage increases the risk of contamination with other cell lines or microorganisms and can promote genetic instability during the course of experiments. The workload associated with the continuous passage and parallel differentiation of multiple iPS lines also increases the chance of human error. A serial approach could separate the expansion and differentiation workload and provide time to perform proper quality control before experiments are performed.

Continuous passage could create an even larger problem for cell therapies. Pluripotent stem cells are banked under cGMP conditions before a battery of expensive tests that verify the integrity and sterility of the cell bank. After validation, banked PSCs are thawed and expanded before initiating differentiation to transform the bank into a clinically relevant cell type. Thawing, expansion and PSC passage creates a major variable and increased complexity during manufacturing. This step can change the timing, yield and quality of PSCs going into the differentiation process.

Therefore, there is a need in the art for methods and compositions for elimination of issues associated with traditional methods of preparing cells for in vitro culture, and for generating high-quality and highly consistent PSCs.

### 3. SUMMARY OF THE INVENTION

The presently disclosed subject matter relates to compositions of ready-to-use cryopreserved cells which can be directly used for downstream applications without after-thaw expansion and/or passage, methods of preparation of such compositions, precursor compositions, and methods of in vitro culturing, or other uses of, cells in said compositions.

The presently disclosed subject matter provides for quality controlled pluripotent stem cells (PSCs) to be used in experiments, and the elimination of batch-to-batch variability through the creation of large batches that can be repeatedly used at different times and places since they are cryopreserved as ready-to-use aliquots.

In accordance with one aspect of the disclosed subject matter, compositions are provided that comprise a frozen population of cells and a cryopreservation medium. In another aspect, the present disclosure provides compositions that include a cell transfected with a heterologous nucleic acid, prepared by transfecting a cell obtained by thawing a frozen population of cells and a cryopreservation medium.

In certain embodiments, the population of frozen cells is a dissociated population of cells. In certain embodiments, the population of frozen cells is in a concentration of at least about 0.5, 1, 5, 10, 20, 30, 40, 50, 60, 70, 80, 90, or 100 million cells/ml. In certain embodiments, the concentration of cells in the frozen population is at least about 1 million cells/ml. In certain embodiments, the concentration of cells in the frozen population is at least about 5 million cells/ml. In certain embodiments, the concentration of cells in the frozen population is at least about 30 million cells/ml. In certain embodiments, the concentration of cells in the frozen population is at least about 50 million cells/ml.

In certain embodiments, the cells are mammalian cells. In certain embodiments, the mammalian cells are pluripotent stem cells (PSCs). In certain embodiments, the pluripotent stem cells are induced pluripotent stem cells (iPSCs) or prepared from embryonic stem cells (ESCs).

Furthermore, the presently disclosed subject matter provides methods for preparing a composition comprising frozen cells, comprising: dissociating a population of cells cultured in a culture medium; suspending the dissociated cells in a cryopreservation medium to form a cell suspension; and freezing the cell suspension to form a composition of frozen cells. Additionally, the present disclosure provides methods for preparing a transfected cell, including (i) preparing a composition comprising frozen cells by a method, such method includes: dissociating a population of cells cultured in a culture medium; suspending the dissociated cells in a cryopreservation medium to form a cell suspension; and freezing the cell suspension to form a composition of frozen cells; and (ii) transfecting a cell from composition (i) with a heterologous nucleic acid.

In certain embodiments, the composition of frozen cells has a concentration of at least about 0.5, 1, 5, 10, 20, 30, 40, 50, 60, 70, 80, 90, or 100 million cells/ml.

In certain embodiments, dissociating a population of cells further includes exposing the population of cells to an effective amount of a cell dissociation solution. In certain embodiments, the cell dissociation solution is selected from the group consisting of enzyme-free cell dissociation solutions and enzyme-containing solutions. In certain embodiments, the enzyme-containing cell dissociation solution comprises one or more enzyme. In certain embodiments, enzyme is selected from the group consisting of Accutase™, collagenase, protease, trypsin and derivatives, papain, hyaluronidase, and DNase. In certain embodiments, the enzyme-free cell dissociation solution comprises a chelating agent. In certain embodiments, the chelating agent is EDTA or other Ca++/Mg−+ free agents used to remove cellular interaction with substrates. In certain embodiments, the culture medium is a feeder-free medium.

In accordance with another aspect of the disclosed subject matter, the presently disclosed subject matter provides in vitro methods for culturing cells, comprising: thawing a composition comprising a population of dissociated frozen cells and a cryopreservation medium; and subjecting the cells to a downstream treatment, wherein the cells are essentially not expanded and/or not passaged before the downstream treatment. In certain embodiments, the composition of frozen cells has a concentration of at least about 0.5, 1, 5, 10, 20, 30, 40, 50, 60, 70, 80, 90, or 100 million cells/ml.

In certain embodiments, the population of cells is subjected to after-thaw expansion. In certain embodiments, the population of cells is expanded for a period of time such that cells in the population undergo up to 1, 2, 3, 4, or 5 rounds of cell division.

In certain embodiments, the population of cells is expanded for a period of time such that cells undergo cell division, wherein said expansion is not exponential expansion In certain embodiments, the population of cells is subjected to after-thaw passage. In certain embodiments, the population of cells is subjected to up to 1, 2, 3, 4, or 5 passages. In certain embodiments, the cells are mammalian cells. In certain embodiments, the mammalian cells are pluripotent stem cells (PSCs). In certain embodiments, the pluripotent stem cells are induced pluripotent stem cells (iPSCs) or prepared from embryonic stem cells (ESCs).

In certain embodiments, the downstream treatment comprises an in vitro method of differentiating the cryopreserved cells. In certain embodiments, the cryopreserved cells are differentiated into a plurality of somatic cells, for example, neuronal cells or precursors thereof, wherein said differentiated cells express detectable levels of one or more markers of said cells. In certain embodiments, the cryopreserved cells are differentiated into neural crest or neural crest derived cells. In certain embodiments, the cryopreserved cells are differentiated into dopamine-producing cells, such as midbrain dopamine cells, or precursor thereof. In certain embodiments, the dopamine precursor cells express detectable levels of forkhead box protein A2 (FOXA2), LIM homeobox transcription factor 1 alpha (LMX1A), and/or tyrosine hydroxylast (TH).

In certain embodiments, the iPSCs are derived from a subject diagnosed with a disease or disorder, for example, a neurodegenerative disease. In certain embodiments, the neurodegenerative disease is Parkinson's disease.

In certain embodiments, the cells described herein are transfected with a nucleic acid prior to cryopreservation, or post-thawing following cryopreservation. In certain embodiments, the nucleic acid is introduced by transfection or nucleofection. In certain embodiments, the cells exhibit increased uptake and/or expression of the nucleic acid compared to cells that have not been subject to cryopreservation. In certain embodiments, the level of expression of the population of dissociated cells is at least about 2 times greater than the level of expression of the population of cells that has not been frozen. In certain embodiments, the level of expression of the population of dissociated cells is at least about 5% greater than the level of expression of the population of cells that has not been frozen.

Accordingly, various embodiments provide for methods of producing transfected cells comprising thawing a composition comprising a population of dissociated frozen cells and a cryopreservation medium; and then transfecting the cells with a nucleic acid of interest, wherein the cells are essentially not expanded and/or essentially not passaged prior to transfection, and wherein the transfection efficiency is substantially increased compared to the transfection efficiency of control cells that had not been cryopreserved prior to transfection, and/or expression of transfected nucleic acid is substantially increased compared with expression of transfected nucleic acid in control cells that had not been cryopreserved prior to transfection. In certain embodiments, compositions of transfected cells prepared by the foregoing method are provided, wherein said cells contain transfected nucleic acid, for example, heterologous nucleic acid which comprises nucleic acid sequence, at least a portion of which is not found in the cells prior to transfection, including sequence altered by insertion, deletion, substitution, or rearrangement.

### 4. BRIEF DESCRIPTION OF THE FIGURES

FIGS. 1A-1L depicts viability, bank consistency, and PSC marker expression of CryoPaused cells. (1A, 1F) Percent viability of fresh (control) and CryoPaused WA09 cells post thaw. Viability was measured on a Nexcelom automated cell counter using Acridine Orange (live) and Propidium Iodide fluorescence (dead) fluorescence (n=15 for CP and n=28 for control). (1B) Viability of cells before freezing (red triangle) and after thawing (black circles) on 13 independent CryoPaused PSC cell banks. (1C-1E) Stem cell marker expression (or spontaneous differentiation, SSEA-1) in control or CryoPaused cells by flow cytometry (1C), values for independent biological replicates shown as mean±SD (1D, n=3) or immunofluorescence (1E, Scale bar=100 μm). (1G) PluriTest assay to assess pluripotency of control and CryoPaused cells. Control (n=2) or CryoPaused (n=2) WA09 cultures were used to obtain RNA before hybridization to a microarray. The array data was processed using the PluriTest algorithm, and each sample is plotted as two related parameters, the Pluripotency Score and the Novelty Score. All four samples passed PluriTest's assessment of pluripotency. Red cloud is representative of samples that passed the PluriTest while the blue cloud are samples that failed PluriTest. (1H) MA plot evaluating the methylation of control and CryoPaused cells. Methylation values for complementary CpG sites (one base apart on opposite strands) were combined to generate CpG-unit methylation. A minimum threshold coverage of 10 reads was used to filter CpG-units resulting in 3,714,418 and 3,408,158 CpG-units for control and CryoPause samples, respectively. Agreement of methylation levels was evaluated by median absolute deviation (MAD), a robust measure of variability insensitive to outliers that estimates the statistical dispersion in methylation levels of the 3,155,482 common CpG-units covered by both samples. (1I) CryoPaused cells are competent to produce teratomas. For all panels, red arrows point to ectoderm, green and blue asterisks denote mesoderm, and black arrows point to endoderm. (Top) Low magnification image showing a hemotoxylin and eosin-stained teratoma section derived from CryoPaused WA09 hESCs. Scale bar=1 mm. (Middle) High magnification image showing regions containing endoderm (goblet cells, black arrows) and mesoderm (green asterisks), scale bar=200 μm. (Bottom) High magnification image showing ectoderm (melanotic neurectoderm, red arrow) and mesoderm (adipocytes, blue asterisk), scale bar=200 μm. (1J) Quantification of DNA damage in control and CryoPaused cells as measured by γH2AX expression on the Operetta High-Content Imager (left) and after treatment with 0.5 μM Camptothecin for 1 hour as a positive control for the assay and to test for vulnerability to DNA damage (right, n=3; values for independent biological replicates shown as mean±SD). (1K) Representative immunofluorescence of γH2AX expression in control and CryoPaused cells with and without treatment with 0.5 μM Camptothecin for 1 hour. (1L) Chromosome analysis was performed on 20 DAPI-banded metaphases, all of which were fully karyotyped. Both control (20/20) and CryoPaused cells (20/20) had a normal 46, XX karyotype. In (1A), (1C) and (1J), Wilcoxin's signed rank test was performed with at least three independent experiments, and no statistical difference (p>0.05) was found between control and CryoPause. See also FIG. 5A.

FIGS. 2A-2M depicts the kinetics and extent of directed differentiation of CryoPaused cells. (2A & 2L) OCT4 and PAX6 expression quantified by flow cytometry during neural induction (n=5; values for independent biological replicates shown as mean±SD). (2B-2C) Representative flow cytometry (2B) and immunofluorescence (2C) at 0, 3, 6 and 9 days after neural induction. (2D) OCT4 and Brachyury expression quantified by flow cytometry during mesendodermal induction (n=3; values for independent biological replicates shown as mean±SD). Representative flow cytometry (2E, 2K) and immunofluorescence (2F) at 0, 1, 2, and 4 days after mesendoderm induction. (2G) Viability of CryoPaused cells post thaw when frozen at 1, 5, 10, 20 and 30 million cells/mL. Percent of viable cells was determined on an automated cell counter with Acridine Orange (live) and Propidium Iodide (dead) fluorescence (n=3; values for independent biological replicates shown as mean±SD). (2H, 2M) Stem cell marker expression (or spontaneous differentiation, SSEA-1) measured by flow cytometry in control and CryoPaused cells expanded in a Cell Factory (n=3; values for independent biological replicates shown as mean±SD in 2H, representative example in 2M). (2I) FOXA2 (red) and TH (green) expression 21 days after midbrain dopamine neuron differentiation of CryoPaused cells. Scale bar=100 μm. (2J) Gene expression of control and CryoPaused WA09 hESC and midbrain dopamine neurons. Samples were normalized to WA09 hES control cells, and the fold change of expression were color coded. Higher levels of expression relative to control hES are shown in red, and lower levels are shown in blue. In (2A), (2D), (2G) and (2H), Wilcoxin's signed rank test was performed with at least three independent experiments, and no statistical difference (p>0.05) was found between control and CryoPause.

FIG. 3A-3E. Genetic modification of CryoPaused cells. A-B) Representative immunofluorescence (A) and flow cytometry (B) of GFP expression in control and CryoPaused cells 24 hours after nucleofection with a GFP plasmid. (C) GFP expression quantified by flow cytometry in control and CryoPaused cells 24 hours after nucleofection with GFP plasmid (n=3; values for independent biological replicates shown as mean±SD). (D) Representative immunofluorescence of GFP expression in CryoPaused cells after transduction with Sendai virus vector containing EmGFP. Individual subclones from initial transduction could be maintained as GFP+ colonies for at least 10 passages. (E) Agarose gel analysis of cleavage products after using a genomic cleavage detection kit with HPRT guide RNA in CryoPaused WA01 iCRISPR cells. + or − indicates with (+) and without (−) detection enzyme. (1) Positive control; (2) iCRISPR cells with Cas9 induced prior to CryoPausing but without guide RNA; (3) iCRISPR cells without Cas9 induction but with HPRT guide RNA; (4) iCRISPR cells with Cas9 induction and HPRT guide RNA; (5) iCRISPR cells (without CryoPausing) with Cas9 induction and HPRT guide RNA. In (C), Wilcoxin's signed rank test was performed with three independent experiments, and no statistical difference (p>0.05) was found between control and CryoPause.

FIGS. 4A-4C depicts genetic modification of CryoPaused cells. GFP expression by flow cytometry (4A) or fluorescence microscopy (4B) 24 and 48 hours post nucleofection with GFP plasmid in control and CryoPaused cells. (4C) The GeneArt Genomic Cleavage Detection Kit positive control with (lane 1) and without (lane 2) enzyme. Dox added to cells before CryoPausing but no guide RNA during nucleofection with (lane 3) and without (lane 4) enzyme. No Dox added to cells before CryoPausing but did receive HPRT guide RNA during nucleofection with (lane 5) and without (lane 6) enzyme. Cells received Dox before CryoPausing and HPRT guide RNA post thaw with (lane 7) and without (lane 8) enzyme. Fresh, control cells that received Dox and HPRT guide RNA (but were never frozen) with (lane 9) and without (lane 10) enzyme.

FIGS. 5A-5F depicts viability of CryoPaused cells with modifications to cryopreservation conditions. Related to FIG. 1. Viability was measured on an automated cell counter using Acridine Orange (live) and Propidium Iodide (dead) fluorescence. (5A) Percent viability of CryoPaused and control cells in WA09, 960.1B, 153.3A, and WA01 iCRISPR cell lines. (5B) Post thaw viability of CryoPaused WA09 cells using either a controlled rate freezer or conventional freezing in FreSR-S or PSC Cryopreservation Kit. (5C) Post thaw viability of CryoPaused WA09 cells using either a controlled rate freezer or conventional freezing (see Methods). (5D) Post thaw viability of CryoPaused WA09 cells grown in either feeder-free (Geltrex™) or feeder-based conditions. “CRF” indicates use of a controlled rate freezer while “−80” indicates storing vials in a cell freezing container at −80° C. for 24 hours before transferring to a liquid nitrogen tank. DMSO indicates using 10% DMSO as the cryomedium. Cells were CryoPaused using either a controlled rate freezer or conventional freezing. (5E) Stem cell marker expression (or spontaneous differentiation, SSEA-1) by flow cytometry in control or CryoPaused cells frozen for over one year. (5F) Teratoma growth rate using control and CryoPaused WA09 hESC.

FIG. 6 is a schematic illustration of traditional method and CryoPause method in preparing and using cryopreserved cells.

FIG. 7 is a different schematic illustration of CryoPause method compared to conventional PSC culture. (Top) The conventional (control) workflow recovers colonies from cryopreservation and expands them over long periods of time, periodically using a portion of the culture for specific applications such as directed differentiation into a cell type of interest. Over time, PSCs might acquire genetic changes, contamination, or changes in the amount of spontaneous differentiation, any of which could affect results. (Bottom) CryoPause expands a large pool of PSCs over the least number of passages possible. The large batch is then dissociated into a single-cell suspension before cryopreservation. The freezing process separates the production of PSCs from their use, allowing time to perform proper quality control and characterization of each bank. It also permits the use of identical cells in multiple experiments, and allows shipping anywhere in the world so that other labs can initiate independent experiments with the exact same starting population of PSCs.

FIGS. 8A-8C depict the expression level distribution of constructs expressed by populations of genetically modified CryoPaused WA09 human embryonic stem cells compared to fresh non-frozen control WA09 cells. (8A) Distribution of GFP expression levels in populations of WA09 stem cells subjected to CryoPause and nucleofected with a 3.4 kb GFP plasmid upon thawing (blue graph, a), fresh non-frozen control WA09 cells nucleofected with the GFP vector (red graph, b), and non-nucleofected WA09 cells (green graph, c), as measured by flow cytometry. (8B) Distribution of mCherry expression levels in populations of WA09 stem cells subjected to CryoPause and nucleofected upon thawing with a 9.3 kb CRISPR/CAS9 plasmid that expresses mCherry, and fresh non-frozen control WA09 cells nucleofected with the mCherry CRISPR/CAS9 plasmid, as measured by flow cytometry. Cells were nucleofected with 2.5 μg, 5 μg, 7.5 μg, 10 μg, or 12.5 μg of plasmid. (8C) Percent of the CryoPaused cells and fresh non-frozen control cells expressing mCherry following nucleofection with 2.5 μg, 5 μg, 7.5 μg, 10 μg, and 12.5 μg of plasmid. A second nucleofection solution, HSC2, was also tested with 2.5 μg of the plasmid under CryoPause and fresh non-frozen conditions, and analyzed for mCherry expression.

### 5. DETAILED DESCRIPTION OF THE INVENTION

The presently disclosed subject matter relates to compositions of ready-to-use cryopreserved cells which can be directly used for downstream application without after-thaw expansion and/or passage, and thus eliminating multiple issues associated with continuous passage, such as contamination and inconsistency of cell quality. The presently disclosed subject matter also relates to methods of preparation of such compositions, precursor compositions, and in vitro methods of using cells in said compositions for downstream applications such as cell differentiation, cell therapy and disease modeling.

For purposes of clarity of disclosure and not by way of limitation, the detailed description is divided into the following subsections:

5.1. Definitions;

5.2. Compositions of ready-to-use cryopreserved cells;

5.3. Methods of preparation of ready-to-use cryopreserved cells; and

5.4. Methods of use of ready-to-use cryopreserved cells.

### 5.1 Definitions

The terms used in this specification generally have their ordinary meanings in the art, within the context of this invention and in the specific context where each term is used. Certain terms are discussed below, or elsewhere in the specification, to provide additional guidance to the practitioner in describing the compositions and methods of the invention and how to make and use them.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude, e.g., within 5-fold, or within 2-fold, of a value.

As used herein, the term “aliquot” means a portion of the total amount of a solution, e.g., a cell suspension solution. An “aliquot of cells” means a portion of the total amount of a cell suspension that is divided from the cell suspension and is stored in a separate container.

As used herein, the term “cell culture” refers to a growth of cells in vitro in an artificial medium for research or medical treatment.

As used herein, the term “adherent cell culture” refers to a cell culture method, in which the cells are grown in a cell culture medium while attached to the bottom of a tissue culture flask or plate.

As used herein, the term “culture medium” refers to a liquid that covers cells in a culture vessel, such as a Petri plate, a multi-well plate, and the like, and contains nutrients to nourish and support the cells. Culture medium may also include growth factors added to produce desired changes in the cells.

As used herein, the term “cell suspension” refers to a solution, in which single cells or small aggregates of cells are suspended in a liquid medium without attaching to the walls of the container. A “single cell suspension” refers to a cell suspension in which single cells are suspended in a liquid medium.

As used herein, the term “cryopreserve” or “cryopreservation” is a process where cells are preserved by cooling to very low temperature.

As used herein, the term “cryopreserve medium” refers to a liquid that is mixed and stored with cryopreserved cells. Cryopreserve media may contain an effective amount of substances that are used to protect cells from freezing damage due to ice formation.

As used herein, the term “expansion” or “expand” refers to an increase in cell number.

As used herein, the term “passage” refers to the process of removing culture medium from a culturing vessel and transferring the cells cultured in the culturing vessel into a fresh culture medium. The passage process enables the further expansion of the cultured cells.

As used herein, the term “in vitro” refers to an artificial environment and to processes or reactions that occur within an artificial environment. In vitro environments exemplified, but are not limited to, petri dishes, conical tubes and cell cultures.

As used herein, the term “in vivo” refers to the natural environment (e.g., an animal or a cell) and to processes or reactions that occur within a natural environment, such as embryonic development, cell differentiation, neural tube formation, etc.

As used herein, the term “differentiation” refers to a process whereby an unspecialized cell, for example, pluripotent stem cells such as embryonic stem cells (ESCs) and/or induced pluripotent stem cells (iPSCs) acquires the features of a specialized cell such as a heart, liver, neuron or muscle cell, or precursors thereof. Differentiation is controlled by the interaction of a cell with the physical and chemical conditions outside the cell, for example through signaling pathways involving proteins embedded in the cell surface that regulate directly or indirectly gene expression.

As used herein, the term “population of cells” or “cell population” refers to a group of cells. In non-limiting examples, a cell population can include at least about 0.1 million, at least about 0.5 million, at least about 1 million, at least about 2 million, at least about 3 million, at least about 4 million, at least about 5 million, at least about 10 million, at least about 20 million, at least about 30 million, at least about 40 million, at least about 50 million, at least about 60 million, at least about 70 million, at least about 80 million, at least about 90 million, at least about 100 million cells, at least about 200 million cells, at least about 500 million cells, at least about 1 billion cells, at least about 1.5 billion cells, at least about 2 billion cells, at least about 2.5 billion cells, at least about 3 billion cells, or values in between. The population may be a pure population comprising one cell type. Alternatively, the population may comprise more than one cell type, for example a mixed cell population.

As used herein, the term “stem cell” refers to a cell with the ability to divide for indefinite periods in culture and to give rise to specialized cells. A human stem cell refers to a stem cell that is from a human.

As used herein, the term “embryonic stem cell” refers to a primitive (undifferentiated) cell that is derived from preimplantation-stage embryo, capable of dividing without differentiating for a prolonged period in culture, and are known to develop into cells and tissues of the three primary germ layers. A human embryonic stem cell refers to an embryonic stem cell that is from a human. As used herein, the term “human embryonic stem cell” or “hESC” refers to a type of pluripotent stem cells derived from early stage human embryos, up to and including the blastocyst stage, that is capable of dividing without differentiating for a prolonged period in culture, and are known to develop into cells and tissues of the three primary germ layers.

As used herein, the term “pluripotent” refers to an ability to develop into the three developmental germ layers of the organism including endoderm, mesoderm, and ectoderm.

As used herein, the term “induced pluripotent stem cell” or “iPSC” refers to a type of pluripotent stem cell (PSC), similar to an embryonic stem cell, formed by the introduction of certain embryonic genes (such as a OCT4, SOX2, and KLF4 transgenes) (see, for example, Takahashi and Yamanaka Cell 126, 663-676 (2006), herein incorporated by reference) into a somatic cell, for examples, CI 4, C72, and the like.

As used herein, the term “pluripotent stem cell line” or “PSC line” refers to a population of pluripotent stem cells which have been cultured under in vitro conditions that allow proliferation without differentiation for up to days, months to years.

An effective amount is an amount that produces a desired effect.

As used herein, the term “inducing differentiation” in reference to a cell refers to changing the default cell type (genotype and/or phenotype) to a non-default cell type (genotype and/or phenotype). Thus, “inducing differentiation in a stem cell” refers to inducing the stem cell (e.g., human stem cell) to divide into progeny cells with characteristics that are different from the stem cell, such as genotype (e.g., change in gene expression as determined by genetic analysis such as a microarray) and/or phenotype (e.g., change in expression of a protein, such as TUJI, DCX, TBR1, REELIN, and FOXG1).

An “individual” or “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

As used herein, the term “disease” refers to any condition or disorder that damages or interferes with the normal function of a cell, tissue, or organ.

As used herein, the term “treating” or “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

### 5.2 Compositions of Ready-to-use Cryopreserved Cells

The presently disclosed subject matter provides for compositions comprising a frozen population of dissociated cells and a cryopreservation medium. The frozen population of cells is ready-to-use, wherein the cells can be used for downstream applications without, or with little, after-thaw expansion and/or passage.

In certain embodiments, the population of dissociated cells comprises single-cell suspensions such that each cell in the population exhibits a reduced level of attachment to other cells compared to a population of cells that is not dissociated. In certain embodiments, the population of dissociated cells comprises a reduced level or amount of cellular aggregates compared to a population of non-dissociated cells. In certain embodiments, the population of dissociated cells comprises cellular aggregates that are smaller in size compared to a population of non-dissociated cells.

In certain embodiments, the population of dissociated cells comprises single-cell suspensions wherein each cell in the population is not attached to other cells in the population, such that the population does not include cell aggregates.

The frozen cells can be stored in a cryopreservation medium with a high concentration, so that a single aliquot of the composition can provide a sufficient number of cells for downstream applications without, or with little, after-thaw expansion. For example, but not by limitation, the concentration of cells in an aliquot may be greater than 1 million cells/ml, or greater than 5 million cells/ml, or greater than 10 million cells/ml, or greater than 20 million cells/ml.

In certain embodiments, the single aliquot of the composition provides a sufficient number of cells for downstream applications without after-thaw expansion.

In certain embodiments, the single aliquot of the composition provides a sufficient number of cells for downstream applications without after-thaw passage.

In certain embodiments, the single aliquot of the composition provides a sufficient number of cells for downstream applications without after-thaw expansion and passage.

In certain embodiments, the single aliquot of the composition provides a sufficient number of cells for downstream applications, or wherein after thawing, the population of cells is expanded for a period of time such that cells in the population undergo a limited amount of cell division, for example, up to 0.5, up to 1, up to 2, up to 3, up to 4, or up to 5 rounds of cell division (0.5 rounds of cell division means culturing for a period of time that is half the doubling time of the cell population).

In certain embodiments, the population of cells is expanded after thawing for a period of time such that cells in the population undergo cell division, wherein said expansion is not exponential expansion.

In certain embodiments, the population of cells is subjected to after-thaw passage. In certain embodiments, the population of cells is subjected to up to 1, up to 2, up to 3, up to 4, or up to 5 passages.

In certain embodiments, the concentration of the cells in the cryopreservation medium is at least about 0.1 million cells/ml, at least about 0.5 million cells/ml, at least about 1 million cells/ml, at least about 5 million cells/ml, at least about 10 million cells/ml, at least about 15 million cells/ml, at least about 20 million cells/ml, at least about 25 million cells/ml, at least about 30 million cells/ml, at least about 35 million cells/ml, at least about 40 million cells/ml, at least about 45 million cells/ml, at least 50 million cells/ml, at least about 55 million cells/ml, at least about 60 million cells/ml, at least about 65 million cells/ml, at least about 70 million cells/ml, at least about 75 million cells/ml, at least about 80 million cells/ml, at least about 85 million cells/ml, at least about 90 million cells/ml, at least about 95 million cells/ml, at least about 100 million cells/ml, at least about 150 million cells/ml, at least about 200 million cells/ml, at least about 250 million cells/ml, at least about 300 million cells/ml, at least about 350 million cells/ml, at least about 400 million cells/ml, at least about 450 million cells/ml, at least about 500 million cells/ml.

In certain embodiments, a single aliquot of the composition has a volume of at least about 1 ml, at least about 2 ml, at least about 3 ml, at least about 4 ml, at least about 5 ml, at least about 6 ml, at least about 7 ml, at least about 8 ml, at least about 9 ml, at least about 10 ml, at least about 15 ml, at least about 20 ml, at least about 25 ml, at least about 30 ml, at least about 35 ml, at least about 40 ml, at least about 45 ml, at least about 50 ml, at least about 55 ml, at least about 60 ml, at least about 65 ml, at least about 70 ml, at least about 75 ml, at least about 80 ml, at least about 85 ml, at least about 90 ml, at least about 95 ml, at least about 100 ml, at least about 150 ml, at least about 200 ml, at least about 250 ml, at least about 300 ml, at least about 350 ml, at least about 400 ml, at least about 450 ml, at least about 500 ml, at least about 1000 ml.

In certain embodiments, the cells are stem cells, for example, pluripotent stem cells (e.g., human pluripotent stem cells). Non-limiting examples of human stem cells include human embryonic stem cells (hESC), human pluripotent stem cell (hPSC), human induced pluripotent stem cells (hiPSC), human parthenogenetic stem cells, primordial germ cell-like pluripotent stem cells, epiblast stem cells, F-class pluripotent stem cells, somatic stem cells, cancer stem cells, or any other cell capable of lineage specific differentiation. In certain embodiments, the human stem cell is a human embryonic stem cell (hESC). In certain embodiments, the human stem cell is a human induced pluripotent stem cell (hiPSC). In certain embodiments, the human stem cell is a human pluripotent stem cell line. Non-limiting examples of human pluripotent stem cell lines include WA09 (H9), 960.1B, 15.3A, and WA01 iCRISPR cell lines. In certain embodiments, the stem cells are non-human stem cells. Non-limiting examples of non-human stem cells non-human primate stem cells, rodent stem cells, dog stem cells, cat stem cells. In certain embodiments, the stem cells are pluripotent stem cells. In certain embodiments, the stem cells are embryonic stem cells. In certain embodiments, the stem cells are induced pluripotent stem cells.

Any type of cell that can grow in an adherent cell culture is suitable for the presently disclosed subject matter. Non-limiting examples of such cells include animal cells, insect cells and plant cells. In certain embodiments, the cells are CHO cells. In certain embodiments, the cells are COS cells. In certain embodiments, the cells are HEK cells, for example, HEK293 cells. In certain embodiments, the cells are HeLa cells. In certain embodiments, the cells are retinal cells.

Any types of cryopreservation medium known in the art can be used with the presently disclosed subject matter. Cryopreservation medium can contain a substance, for example a cryoprotective agent that can protect cells from freezing damage due to ice formation. Non-limiting examples of cryoprotective agents include glycols, such as ethylene glycol, propylene glycol and glycerol, dimethyl sulfoxide (DMSO), and trehalos. Non-limiting examples of cryopreservation media include FreSR™-S, PSC Cryopreservation medium (ThermoFisher Scientific), Stem-CellBanker GMP, Essential 8™ (ThermoFisher Scientific) with 10% DMSO, CryoStor® Freeze Media (Biolife), and CryoStem cryopreservation medium (Stemgent). In certain embodiments, the cryopreservation medium is FreSR™-S.

The composition comprising the cells and cryopreservation medium can be aliquoted and stored in any types of storage container or vessel known in the art that are suitable for cryopreservation. Non-limiting examples of cryopreservation containers include vials, plastic bags, tubes, and boxes, such as for example, glass vials (for example, but not limited to, flint glass vials), ampoules, plastic flexible containers, for example, but not limited to, PVC (polyvinyl chloride) containers, CZ resin containers, poly propylene containers and syringes, and glass syringes.

### 5.3 Methods of Preparation of Ready-to-use Cryopreserved Cells

The presently disclosed subject matter provides for methods of preparing a composition comprising a population of frozen dissociated cells. The composition can be directly used for downstream applications without, or with little, after-thaw expansion and/or passage.

In certain non-limiting embodiments, the method of preparing a population of frozen dissociated cells comprises: dissociating a population of cells cultured in a culture medium; suspending the dissociated cells in a cryopreservation medium to form a cell suspension; and freezing the cell suspension to form a composition of frozen cells. In certain embodiments, the cells are expanded prior to freezing.

In certain embodiments, at least 60, 65, 70, 75, 80, 85, 90, or 95% of the cells are viable after thawing.

In certain embodiments, the composition of frozen dissociated cells has a concentration of at least about 0.1 million cells/ml, at least about 0.5 million cells/ml, at least about 1 million cells/ml, at least about 5 million cells/ml, at least about 10 million cells/ml, at least about 15 million cells/ml, at least about 20 million cells/ml, at least about 25 million cells/ml, at least about 30 million cells/ml, at least about 35 million cells/ml, at least about 40 million cells/ml, at least about 45 million cells/ml, at least 50 million cells/ml, at least about 55 million cells/ml, at least about 60 million cells/ml, at least about 65 million cells/ml, at least about 70 million cells/ml, at least about 75 million cells/ml, at least about 80 million cells/ml, at least about 85 million cells/ml, at least about 90 million cells/ml, at least about 95 million cells/ml, at least about 100 million cells/ml, at least about 150 million cells/ml, at least about 200 million cells/ml, at least about 250 million cells/ml, at least about 300 million cells/ml, at least about 350 million cells/ml, at least about 400 million cells/ml, at least about 450 million cells/ml, at least about 500 million cells/ml.

In certain embodiments, a single aliquot of the composition has a volume of at least about 1 ml, at least about 2 ml, at least about 3 ml, at least about 4 ml, at least about 5 ml, at least about 6 ml, at least about 7 ml, at least about 8 ml, at least about 9 ml, at least about 10 ml, at least about 15 ml, at least about 20 ml, at least about 25 ml, at least about 30 ml, at least about 35 ml, at least about 40 ml, at least about 45 ml, at least about 50 ml, at least about 55 ml, at least about 60 ml, at least about 65 ml, at least about 70 ml, at least about 75 ml, at least about 80 ml, at least about 85 ml, at least about 90 ml, at least about 95 ml, at least about 100 ml, at least about 150 ml, at least about 200 ml, at least about 250 ml, at least about 300 ml, at least about 350 ml, at least about 400 ml, at least about 450 ml, at least about 500 ml, at least about 1000 ml.

In certain embodiments, the cells are cultured in a culture medium before subject to dissociation. Any types of culture media that are suitable for culturing cells can be used with the presently disclosed subject matter. In certain embodiments, the culture medium is a feeder-free culture medium. In certain embodiments, the culture medium is a feeder medium. In certain embodiments, the medium is an Essential 8™ medium.

Cells cultured in a culture medium are dissociated from a culturing surface, such as culturing plates or flasks, using a cell dissociation solution. Dissociation can also assist separating cells from each other to form a single-cell suspension. Any types of agents or solutions known in the art that are suitable for dissociating cells from cell attachment can be used with the presently disclosed subject matter as a cell dissociation solution. The cell dissociation solution can contain enzymes having a proteolytic and/or collagenolytic function. Non-limiting examples of such enzymes include Accutase™, collagenase, protease, trypsin and derivatives, papain. The cell dissociation solution can also contain other enzymes, such as hyaluronidase and DNase. Hyaluronidase is a family of enzymes that can catalyze the degradation of hyaluronic acid. DNase is a family of enzymes that can digest nucleic acids that leak into the dissociation medium. Non-limiting examples of such enzyme-containing solutions include trypsin buffer, trpsin-EDTA buffer, Accutase, Detachin™ Cell Detachment Solution (Genlantis), and Accumax. The cell dissociation solution can be enzyme-free. In certain embodiments, the enzyme-free cell dissociation solution can contain a chelating agent to chelate free calcium and magnesium ions in the solution, and thus dissociate cells. Non-limiting examples of chelating agents are EDTA or other Ca++/Mg++ free agents used to remove cellular interaction with substrates, 1,1-bis(diphenylphosphino)ethylene. Non-limiting examples of such enzyme-free solutions include Gentle Cell Dissociation Reagent (GCDR, STEMCELL Technologies); Cell Dissociation Buffer, enzyme-free, Hanks' Balanced Salt Solution (ThermoFisher); and Cell Dissociation Buffer, enzyme-free, PBS (ThermoFisher); or EDTA. In certain embodiments, cells are dissociated using Accutase.

In certain embodiments, the dissociated population of cells is washed before they are suspended in cryopreservation medium. The dissociated population of cells can be washed with any solution known in the art that suitable for washing cells. In certain embodiments, the dissociated population of cells is washed in Essential 8™ medium.

The cell suspension can be aliquoted and stored in any types of storage contains known in the art that are suitable for cryopreservation. Non-limiting examples of cryopreservation containers and vessels are described supra.

Any freezing methods known in the art that are suitable for freezing a cell suspension to cryopreserve the cells can be used with the presently disclosed subject matter, for example, a controlled-rate freezer program.

In certain embodiments, the freezing method comprises using a controlled-rate freezer programmed with the following program:

Step 1: wait at 4° C.;

Step 2: 1.2° C./min (sample) to −4° C.;

Step 3: 25° C./min (chamber) to −40° C.;

Step 4: 10° C./min (chamber) to −12° C.;

Step 5: 1.0° C./min (chamber) to −40° C.;

Step 6: 10° C./min (chamber) to −90° C.; and

Step 7: wait at −90° C.

In certain embodiments, the frozen aliquot of cells can be transferred to a liquid nitrogen tank after controlled rate freezer reached −90° C.

In certain embodiments, the freezing method is a conventional slow-rate cooling method.

### 5.4 Methods of Use of Ready-to-use Cryopreserved Cells

The presently disclosed subject matter also provides for in vitro methods of culturing, or otherwise using, a composition comprising a population of frozen dissociated cells and a cryopreservation medium. In certain embodiments, the in vitro method comprises subjecting the composition to a downstream treatment, wherein the cells are not expanded and/or passaged before the downstream treatment.

In certain embodiments, the composition comprising a population of frozen dissociated cells contains an adequate number of cells for downstream treatment. For example, a single aliquot of the composition can comprise a sufficient number of cells for a downstream treatment.

In certain embodiments, the single aliquot of the composition provides a sufficient number of cells for downstream applications, wherein after thawing, the population of cells is expanded for a period of time such that cells in the population undergo cell division, for example, at least 1, 2, 3, 4, or 5 rounds of cell division. In certain non-limiting embodiments, the population of cells is maintained in culture (optionally with addition of cell culture medium), prior to use, for a period of time sufficient for up to 2 cell divisions, or for a period of time sufficient for up to 5 cell divisions.

In certain non-limiting embodiments, the population of cells is maintained in culture (optionally with addition of cell culture medium), prior to use, for a period of time up to about 1, 5, 10, 15, 20, 25, or 30 hours, for example, after the population of cells is thawed.

In certain non-limiting embodiments, the population of cells is maintained in culture (optionally with addition of cell culture medium), prior to use, for a period of time up to about 24 hours, for example, after the population of cells is thawed.

In certain embodiments, the population of cells is expanded after thawing for a period of time such that cells in the population undergo cell division, wherein said expansion is not exponential expansion.

In certain embodiments, the population of cells is subjected to after-thaw passage. In certain embodiments, the population of cells is subjected to up to 1, 2, 3, 4, or 5 passages.

In certain embodiments, the population of frozen dissociated cells comprises human iPSCs or human ESCs. In certain embodiments, the downstream treatment comprises a method of differentiating the population of cells into plurality of somatic cells, for example, neurons or progenitors thereof. In certain embodiments, said differentiated cells can be comprised in a therapeutic composition, for example a pharmaceutical composition, for therapeutic use. In certain embodiments, the differentiated cells can be used for modeling a disease in vitro. For example, methods of differentiating pluripotent stem cells are disclosed in International publication Nos. WO/2010/096496, WO/2011/149762, WO/2013/067362, WO/2016/19666, WO/2014/176606, WO/2015/077648, and International Application Nos. PCT/US16/068430 filed Dec. 22, 2016, PCT/US17/016723 filed Feb. 6, 2017, and PCT/US17/015480 filed Jan. 27, 2017, the contents of each of which are incorporated by reference in their entireties herein for all purposes.

In certain embodiments, the population of cells described herein is differentiated into a plurality of dopamine cells, for example, midbrain dopamine cells, or precursor thereof. In certain embodiments, the dopamine precursor cells express a detectable level of forkhead box protein A2 (FOXA2), LIM homeobox transcription factor 1 alpha (LMX1A), and/or tyrosine hydroxylast (TH). In certain embodiments, the population of frozen dissociated cells comprises iPSCs that are derived from a subject diagnosed with, or at risk of having, a neurodegenerative disease, e.g., Parkinson's disease.

In certain embodiments, the cells described herein are transfected with exogenous nucleic acid, for example, subjected to nucleofection, electroporation, lipid/liposome-based, calcium phosphate induced, or other methods that move exogenous genetic material into mammalian cells. In certain embodiments, the cells described herein exhibit an increased level of nucleic acid uptake and/or expression of the nucleic acid following transfection, compared to control cells that have not been frozen. In certain embodiments, the increased level of expression by the cells described herein is at least about 2, 3, 4, 5, 6, 7, 8, 9, 10, 15, 20, or 25 times greater that the level of expression by the non-frozen control cells.

In certain embodiments, the increased level of expression by the cells described herein is at least about 5, 10, 15, 20, 25, 30, 35, 40, 45, 50, 55, 60, 65, 70, or 75% greater that the level of expression by the non-frozen control cells.

In certain embodiments, the increased level of expression refers to the amount of cells in a population expressing the nucleic acid.

In certain embodiments, the increased level of expression refers to the intensity or amount of mRNA or protein expressed by one or more cells, or a population of cells.

In certain embodiments, the cells are transfected with nucleic acid prior to cryopreservation.

In certain embodiments, the cells are transfected with nucleic acid following cryopreservation, for example, after thawing. In certain embodiments, the cells are transfected at least about 0.1, 0.2, 0.5, 1, 2, 3, 4, 5, 6, 7, 8, 9, 10, 11, 12, 13, 14, 15, 16, 17, 18, 19, 20, 21, 22, 23, or 24 hours post-thawing following cryopreservation.

In certain embodiments, the nucleic acid comprises nucleic acid molecules for use in CRISPR gene editing, for example, nucleic acids encoding Cas9 protein, CPF1 protein, and/or guide RNA for a predetermined target (see, e.g., Cong et al., Science February 15; 339 (6121):819-23 (2013); Hsu et al., Cell. 2014 June 5; 157 (6):1262-78; Ran et al., Nature Protocols November; 8 (11):2281-308. (2013); Gilbert et al., Cell 2013 July 18; 154 (2):442-51; Staahl et al., Nat Biotechnol. 2017 May; 35 (5):431-434; and Mali et al., Science. 2013 February 15; 339 (6121):823-6, the contents of each of which are incorporated by reference in their entireties).

In certain embodiments, compositions of the transfected cells are provided. In certain embodiments, kits are provided comprising materials for practicing one or more of the above methods. Such kits may comprise, for example, cryopreserved cells and a nucleic acid encoding a molecule for CRISP gene editing, or an expression cassette comprising a promoter sequence, etc.

### 6. EXAMPLES

The presently disclosed subject matter will be better understood by reference to the following Example, which is provided as exemplary of the presently disclosed subject matter, and not by way of limitation.

### Example 1: Cryopreserved Dissociated Pluripotent Stem Cells for Use Post-thaw Without Passage

### Summary

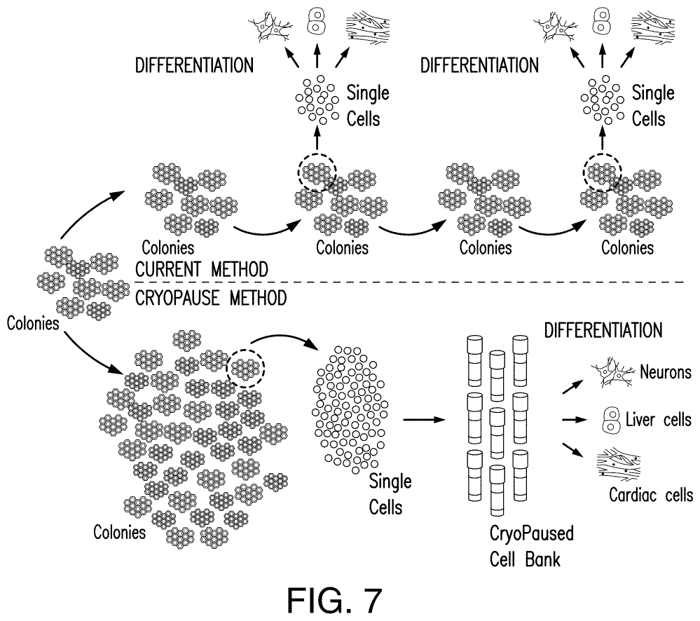

Human pluripotent stem cells (PSCs) provide an unlimited cell source for cell replacement therapies and disease modeling. Despite their enormous power, technical aspects have hampered reproducibility. What is described here is a simple modification of PSC workflows that eliminates a major variable for nearly all PSC experiments: the quality and quantity of the PSC starting material. Most labs serially passage PSCs and use small quantities after expansion but the “just in time” nature of these experiments means that quality control rarely happens before use. Lack of quality control means that PSC quality, sterility and genetic integrity could be compromised which creates a confounding factor in results obtained. The method disclosed here, called CryoPause (FIG. 7), dissociates cultures of PSCs into single cells and banks the PSCs as single-use, cryopreserved vials that can be thawed and immediately used in experiments, such as differentiation.

Here cells cryopreserved using CryoPause were tested against cells cultured in traditional method. It is shown here that no differences in short-term viability or pluripotency. Further CryoPause showed no loss in differentiation efficiency in mesoderm and neural differentiation. Viability post thaw was routinely greater than 90% and it was found no significant difference in stem cell marker expression post thaw. It was found that CryoPaused cells can be thawed and directly differentiated or genetically modified without expansion. The current data provides a simple, new way for any lab to use PSCs that will increase reproducibility, help synchronize different PSC lines before differentiation and eliminate the possibility of genetic instability and contamination during expansion of PSCs for cell therapy and disease modeling applications.

CryoPause allows multiple experiments to be repeated from the same, quality-controlled PSCs and eliminates the possibility of genetic instability and contamination during the expansion of PSCs for both cell therapy and disease modeling applications. In addition, CryoPause allows one to perform multiple differentiations or gene modification experiments at different points in time from an identical starting PSC population. It enables geographically separated laboratories to experiment on an identical starting PSC population. Aside from the consistency, each bank can be pre-validated before use to reduce the possibility that high levels of spontaneous differentiation, contamination or genetic integrity compromised the experiment. Any lab can implement CryoPause to increase reproducibility in both disease modeling and cell therapy applications.

### Results

The current study shows that there is no difference in viability, pluripotency, and differentiation capability, but a slight reduction in plating efficiency was observed. The CryoPause can be scaled up. For example, a 280 million cell bank from a 4-tier cell factory can be constructed. In the present example, cells were frozen at a concentration as high as 30 million cells per vial with no significant decrease in post-thaw viability.

### Developing CryoPause Conditions

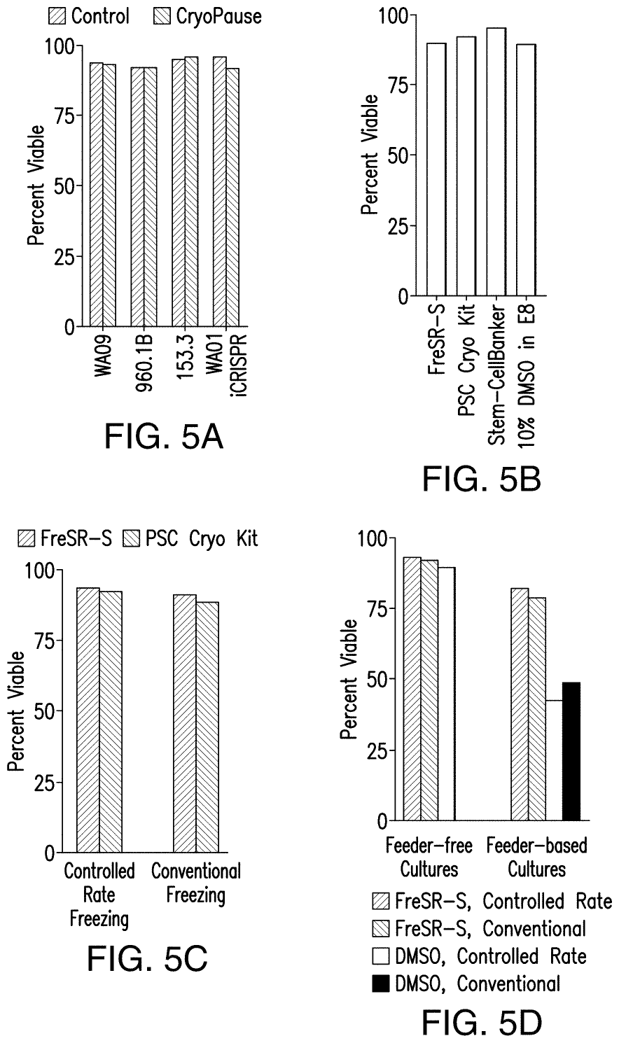

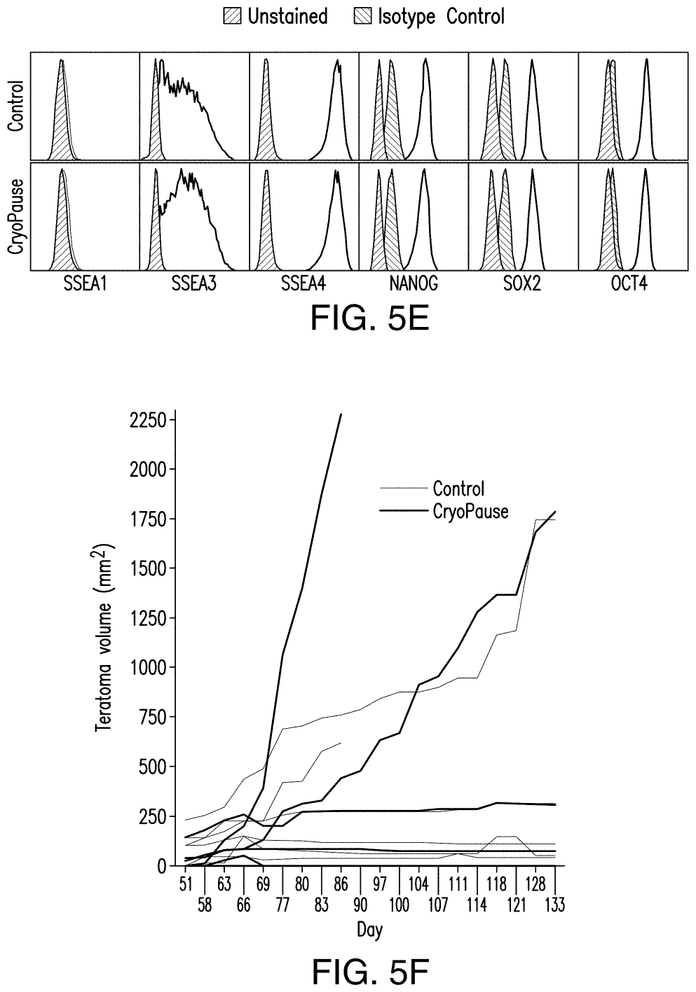

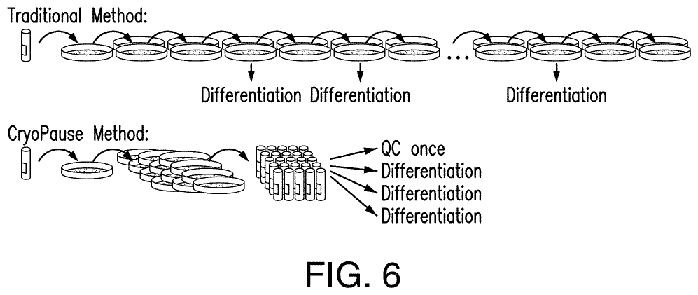

Whether post-thaw expansion could be eliminated to improve the reproducibility of stem cell experiments was determined. Bypassing recovery and expansion likely requires high viability post thaw to be practical. A clinical paradigm using WA09 (H9) feeder-free cultures in Essential 8™ medium (E8; Chen et al. 2011) was used. WA09 cells were treated with Accutase to create a single-cell suspension before washing and resuspending in FreSR™-S, a commercial medium designed for monodispersed human pluripotent stem cell cryopreservation. Cells were cryopreserved in a controlled rate freezer using a standard program before long-term storage in liquid nitrogen as “ready-to-use” aliquots (see Materials and Methods). The initial experiments demonstrated surprisingly high post-thaw viability. The effect of cell line, cryopreservation media and the contribution of the controlled rate freezer to viability post thaw was explored and found no obvious dependence on any of these variables when using feeder-free culture in E8 medium. Different PSC lines (FIG. 5A), different cryopreservation media (FIG. 5B) and conventional slow-rate cooling (FIG. 5C) had no appreciable difference in efficiency. The main factor appeared to be the initial culture conditions since WA09 cells expanded using traditional feeder-based methods had lower viability, although this viability decrease was mitigated in FreSR™-S (FIG. 5D).

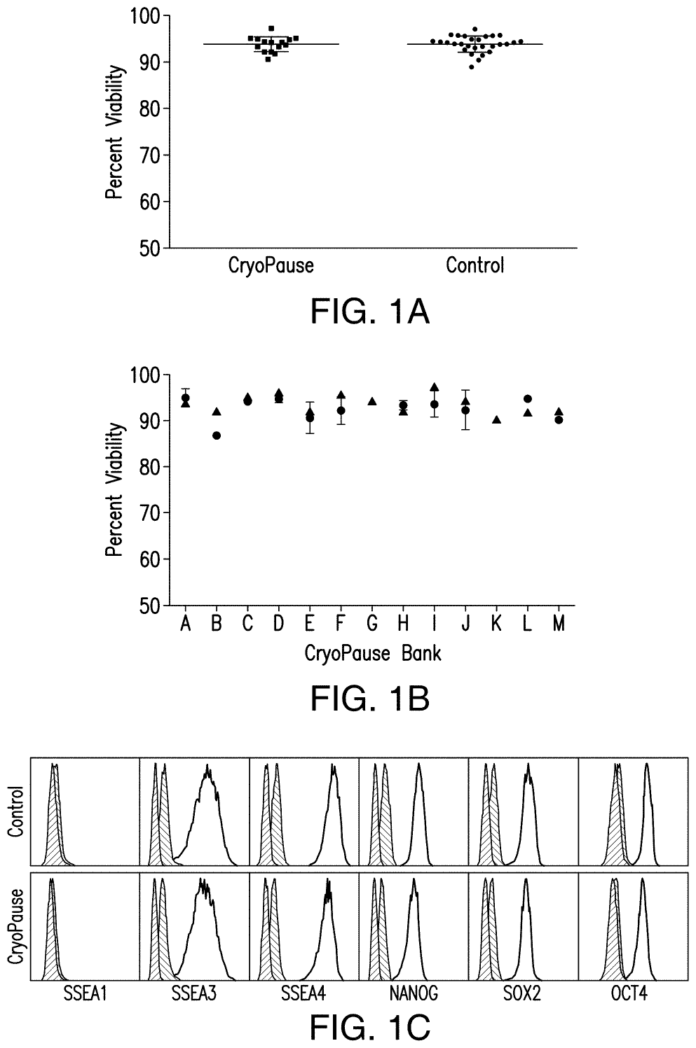

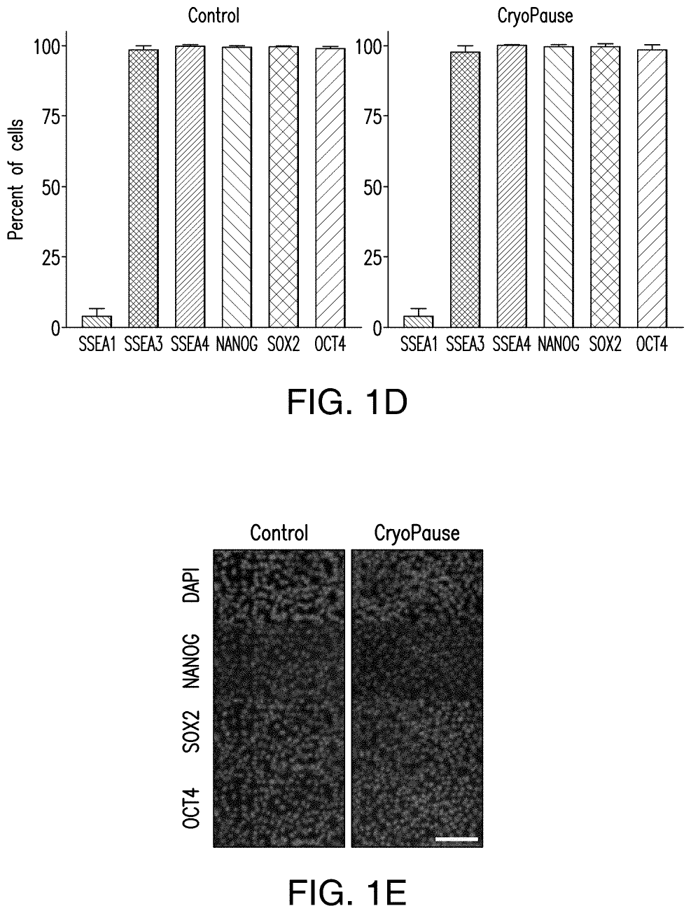

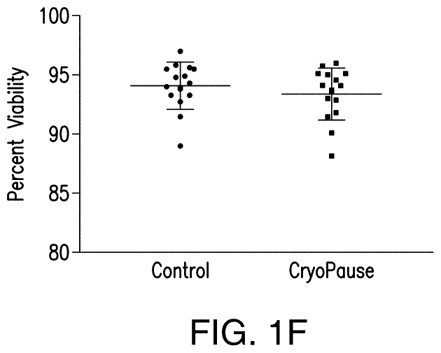

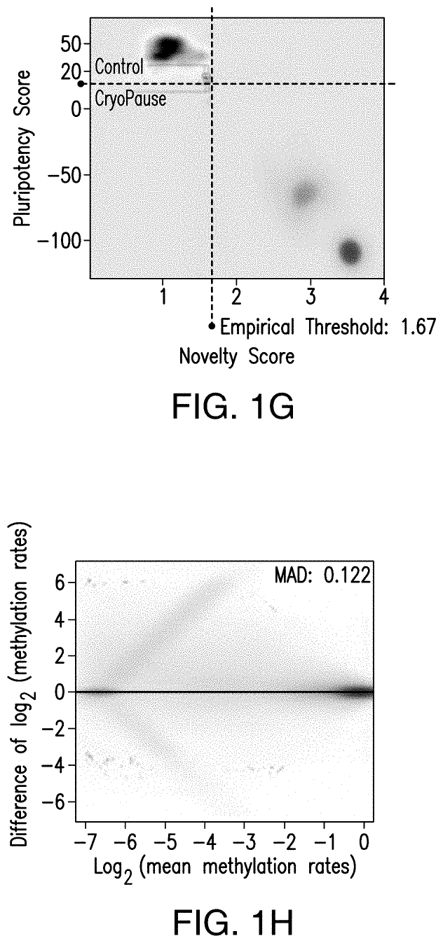

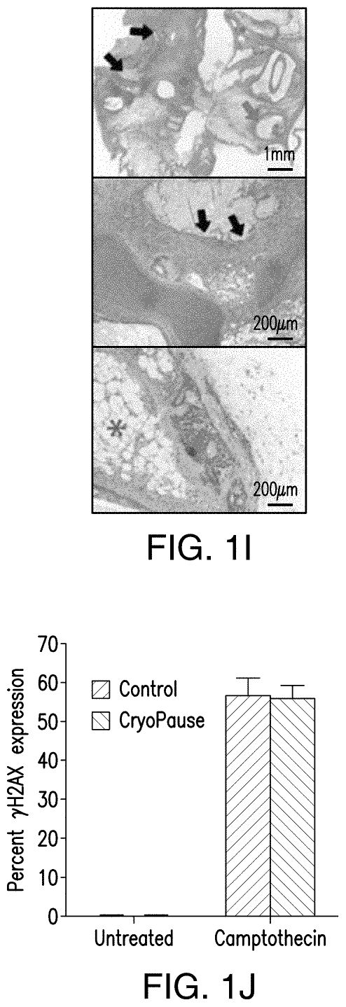

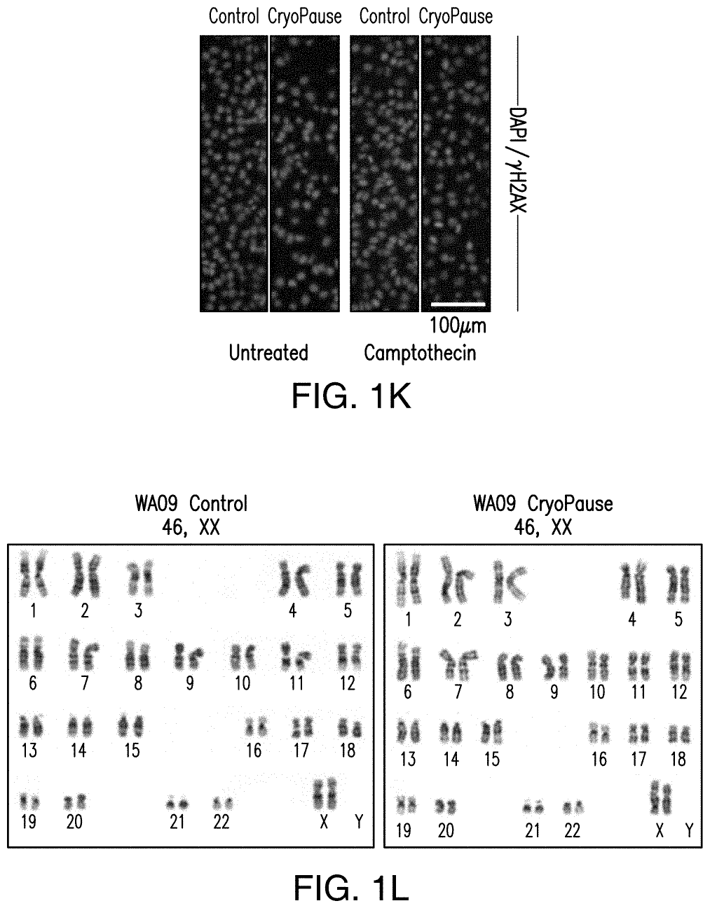

For the balance of the experiments, the baseline condition was WA09 CryoPaused in FreSR™-S in a controlled rate freezer unless otherwise noted. This condition routinely yielded post thaw viability just a few percent lower than control WA09 cells that were not frozen when measured with an unbiased, automated cell counter using acridine orange/propidium iodide (AOPI) (FIGS. 1A & 1F). After the creation of multiple banks, it was learned that the viability post thaw was only limited by the input culture's viability CryoPausing (FIG. 1B). No loss in viability or pluripotency was observed for up to a year of storage in liquid nitrogen, the longest time point tested to date (FIG. 5E). Despite exceptional post-thaw viability, there was a decrease in plating efficiency after 24 hours of culture (data not shown) so it was plated more CryoPaused cells to compensate for this difference in plating efficiency (400,000 cells/cm² in CryoPaused to 200,000 cells/cm² in fresh controls).

### Validating CryoPause Cells

To assess the health of CryoPaused cells, PSC markers were examined one day after plating when differentiation would normally be induced. No obvious difference in the pluripotent stem cell markers SSEA3, SSEA4, OCT4, SOX2 and NANOG could be measured with flow cytometry between CryoPaused and fresh control cells (FIGS. 1C & 1D). There was no increase in the spontaneous differentiation marker SSEA1 (FIGS. 1C & 1D). Immunofluorescence analysis independently confirmed the flow results with no discernable difference between the two populations (FIG. 1E).

To perform a more comprehensive examination of the pluripotent features in CryoPaused PSCs, PluriTest was used to compare control and CryoPaused cultures (Müller et al. 2011; Williams et al. 2011; Müller et al. 2012). PluriTest is based on whole-genome transcriptional profiles and allows for the reliable assessment of pluripotency in undifferentiated stem cell cultures. Briefly, PluriTest analyzes the expression of a large number of pluripotency associated transcripts with a “Pluripotency Score” and tests for the conformity of a tested sample with global transcriptional patterns typically observed in genetically and epigenetically normal human pluripotent stem cells with a second metric, termed “Novelty Score”. With PluriTest, it was found that both control as well as CryoPaused WA09 samples pass the empirically defined Pluripotency and Novelty Score thresholds and demonstrate fit to the Novelty one-class classifier model, which indicates that both sample groups show highly similar gene expression patterns to those observed in well characterized hESC and hiPSC cell lines (FIG. 1G). The epigenetic landscape of CryoPause cells was also surveyed through reduced representation bisulfide sequencing (RRBS) and found strong agreement in methylation levels of common CpG-units between control and CryoPause cells (FIG. 1H; MAD=0.122), which is comparable to the concordant methylation levels observed among technical replicates (Kacmarczyk et al. 2016).

Another measure of pluripotency is the creation of teratomas. Control and CryoPause cultures created teratomas with roughly the same size and kinetics (FIG. 5F). Hemotoxylin and eosin-stained sections of CryoPause teratomas showed derivatives of all 3 germ layers (FIG. 1I: endoderm [black arrows], mesoderm [green and blue asterisks] and ectoderm [red arrows]). Collectively, these data demonstrate that there is no difference in the expression of key pluripotency markers by flow cytometry, PluriTest, CpG methylation, and teratoma formation using CryoPause cells directly post thaw.

To assess the proportion of cells with double-stranded DNA breaks in their genomes, quantitative immunofluorescence of nuclear gH2AX on a high content microscope was performed (FIGS. 1J & 1K; for review, see Redon et al. 2002). There was no statistical difference in the percentage of cells expressing gH2AX. To validate the assay and see if CryoPause cells were more sensitive to DNA damaging agents, Camptothecin, a cytotoxic quinolone alkaloid, was added. There was no statistical difference in the percentage of cells expressing γH2AX after Camptothetin exposure. Finally, we analyzed karyotypes on CryoPause cells post thaw to test for global genetic abnormalities. Normal karyotypes were found in control (20/20 metaphase spreads) and CryoPause cultures (20/20 metaphase spreads: FIG. 1L).

Directed Differentiation and Cell Therapies

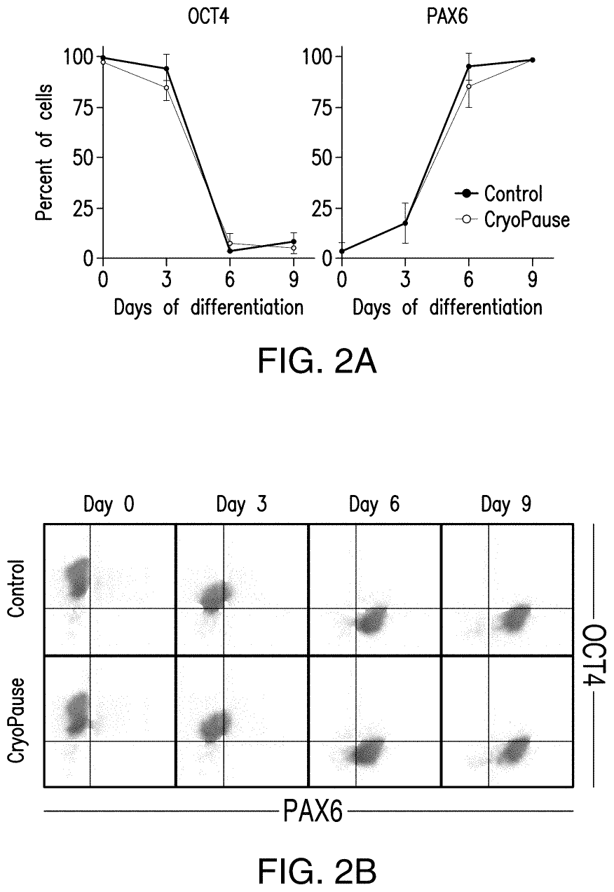

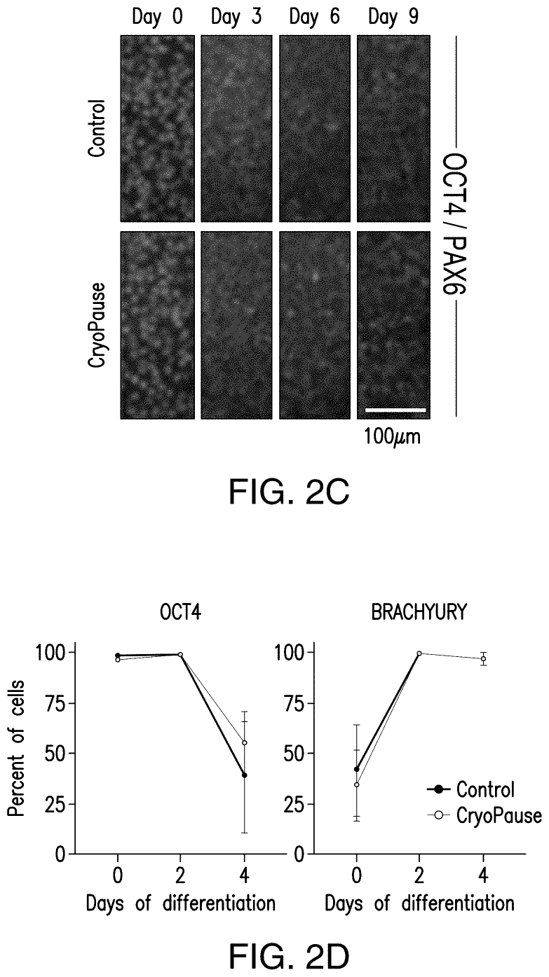

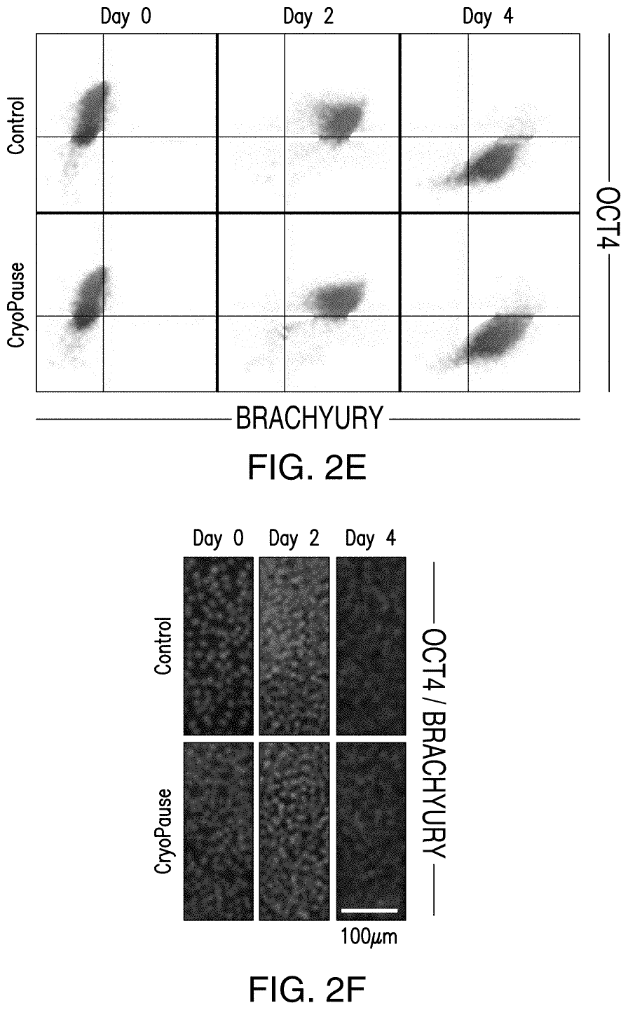

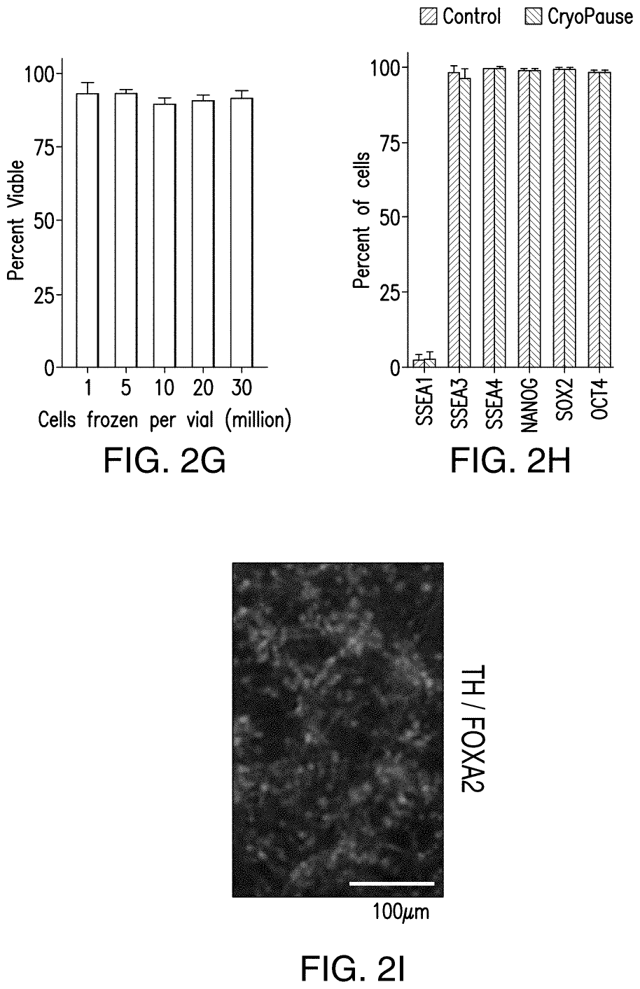

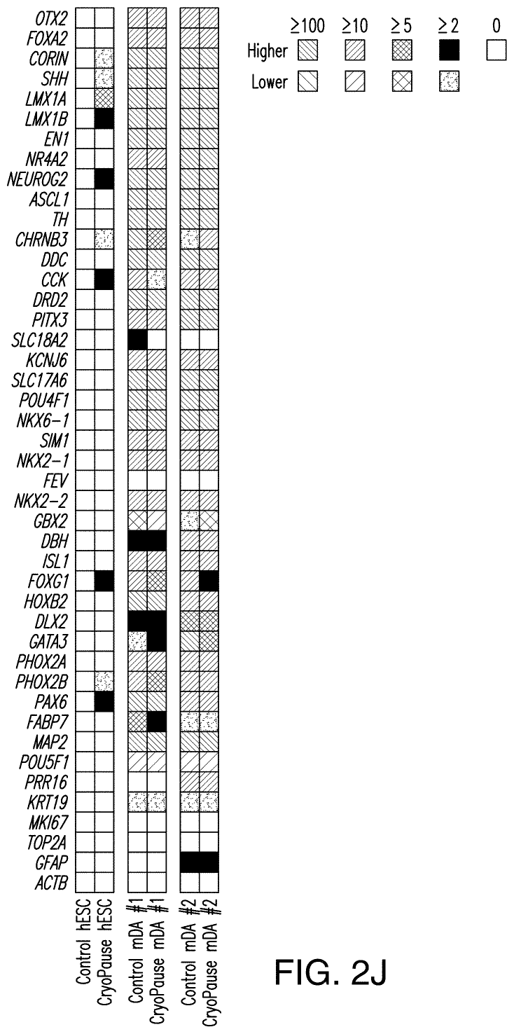

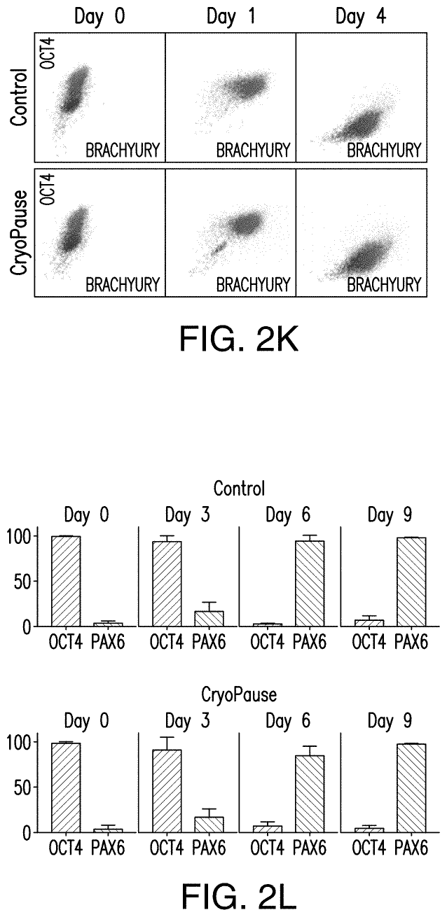

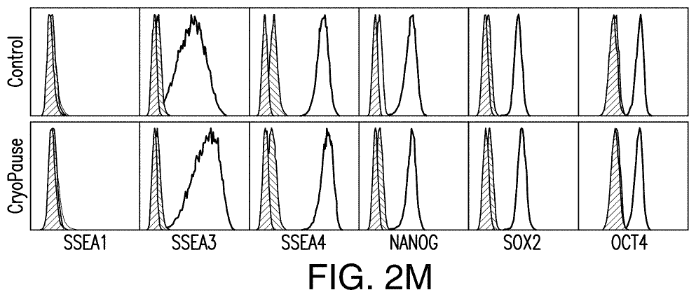

Because little difference between CryoPaused and control cells could be found, the in vitro differentiation capacity of CryoPaused cells was assessed. Control and CryoPaused cells were exposed to dual SMAD inhibition to direct WA09 hESCs to a neural fate (Chambers et al. 2009). OCT4 and PAX6 were measured over time to assess the efficiency and kinetics of neural differentiation; there was no difference in the timing nor the extent of neural induction (FIGS. 2A-2C, and 2K). CryoPaused cells directed to mesendoderm fates were also indistinguishable in the extent and kinetics of differentiation (FIGS. 2D-2F, and 2L). Both of these cell fates are relatively immature and therefore easier to make, so attempts were made to make midbrain dopamine neurons from CryoPaused cells (Barker et. al., 2015). WA09 CryoPaused cells efficiently created FOXA2/TH double positive, post-mitotic midbrain dopamine neurons from a clinically compatible “standard operating procedure” and display a similar gene expression profile as neurons derived from control cells (FIGS. 2I & 2J).

The manufacturer specifies a cell density of 1 million cells/mL when cryopreserving with FreSR™-S. This density is adequate for smaller scale experiments but becomes limiting for applications requiring large cells numbers such as cell therapies where billions of cells are needed per experiment. Therefore, it was tested whether increasing the cell density during cryopreservation would adversely affect viability or plating efficiency. It was found no obvious difference in the viability (FIG. 2G) or plating efficiency (data not shown) when freezing cells up to a density of 30 million/mL, the highest tested. High-density preparations should provide a reasonable workflow for most therapeutic applications that usually require billions of input PSCs before manufacturing.

The strength of CryoPause is the creation of large, characterized, ready-to-use cell banks. While there are many advantages to this strategy, the disadvantage is that it requires all PSC expansion to be performed all at once prior to cryopreservation and quality control. In an effort to scale expansion, “cell factories” was used: conjoined flasks that allow easy feeding of all layers at one time that can easily be adapted to become a closed system with automation. PSCs were expanded using the 4-layer flask size (2528 cm2, or around 44×6-well plates), and had an average yield of ˜250 million cells per factory (n=3). A complication of growth in such “factories” is that the morphology is difficult to monitor during expansion since the layered flasks do not permit direct microscopic observation. To verify that CryoPause banks were expanded correctly, PSC markers were tested and found that factory-expanded cells had equivalent marker expression (FIGS. 2H & 2M) and were capable of differentiation (data not shown).

### Genetic Modification

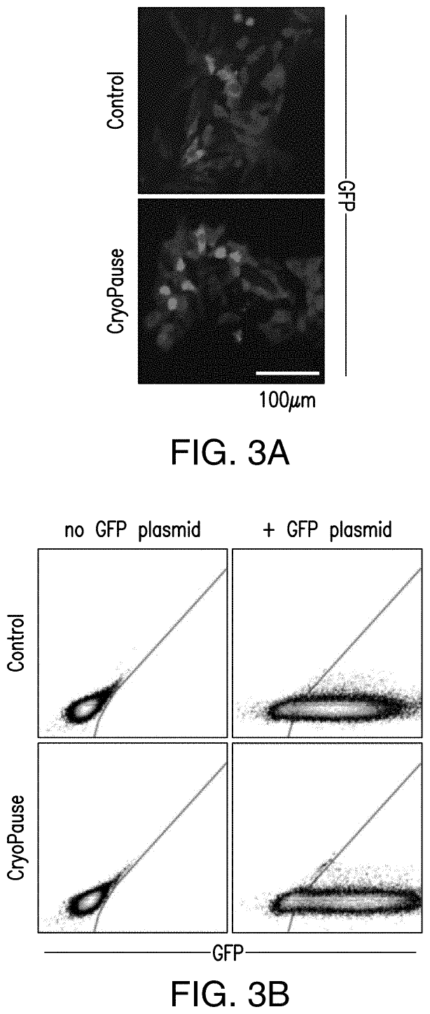

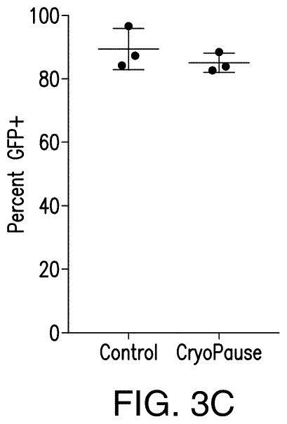

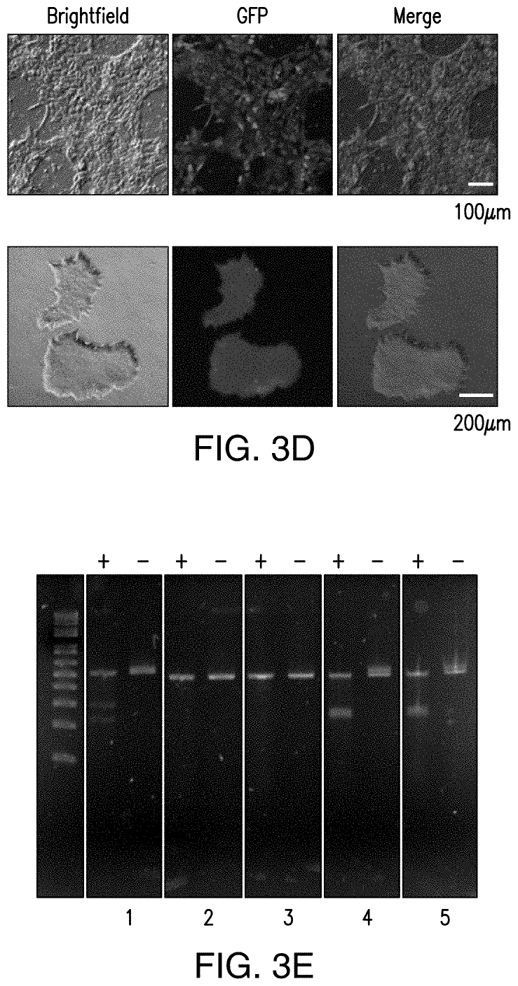

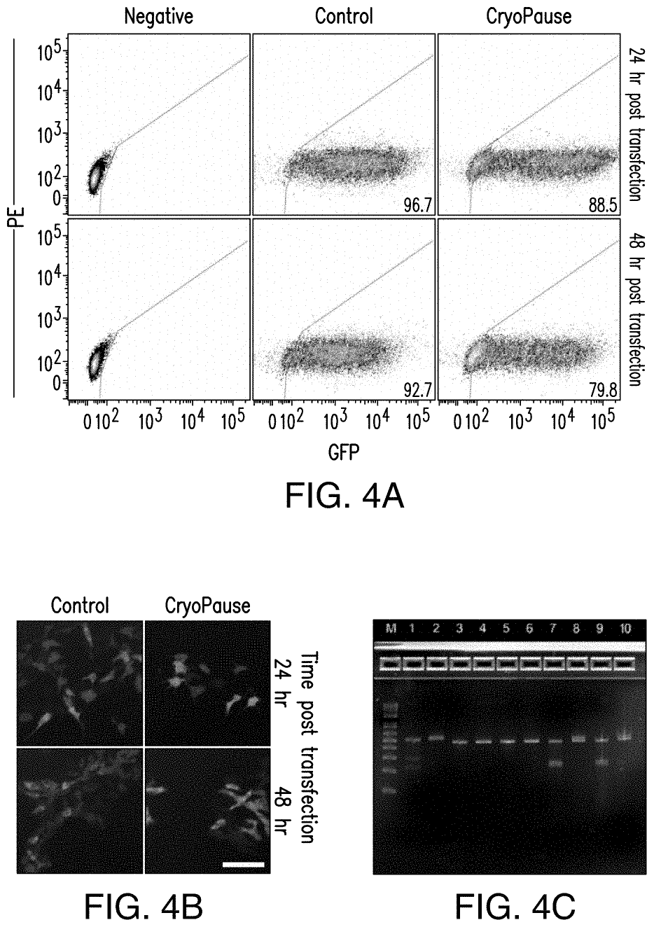

Because CryoPaused cells behave normally 24 hours post thaw, whether the cells could be genetically manipulated immediately post thaw was investigated. The nucleofection efficiency of CryoPaused WA09 cells immediately post thaw were compared to fresh control cells. Twenty-four hours post nucleofection with a GFP plasmid, fluorescent microscopy revealed GFP+ cells in both conditions and flow cytometry quantitation revealed that >85% expressed GFP (FIGS. 3A-3C and 4A-4B). CryoPaused cells that were immediately thawed could also be successfully transduced by a Sendai viral vector expressing EmGFP, as shown by fluoresce microscopy 24 hours post transduction (FIG. 3D). CP-derived, Sendai transduced subclones could be propagated for at least 15 passages, the longest tested.

Nucleofection success suggested that genome modification upon thawing might work. To test targeted genome modification, an iCRISPR system was used, PSCs that contain inducible Cas9 engineered into the AAVS1 locus (Gonzalez et al. 2014). WA01 (H1) iCRISPR cells were expanded then treated with doxycycline 24 hours before CryoPausing: this created CryoPaused cells that pre-expressed Cas9 before freezing. Once thawed, HPRT guide RNA was nucleofected into these cells before genomic analysis. iCRISPR cells that were CryoPaused with Cas9 expressed were thawed and nucleofected with HPRT guide RNA immediately post thaw. There were no obvious differences in the efficiency of HPRT targeted mutations between CryoPaused and control iCRISPR WA01 (H1) cells (FIGS. 3E and 4C).

### Discussion

A new method (CryoPause) is described that eliminates a critical variable for most pluripotent stem cell-based applications: the nature of pluripotent cells before differentiation or genomic modification. It is commonly accepted that cryopreserved hPSCs require recovery, expansion and passage before use. The present experiment demonstrated that dissociated human pluripotent stem cells can be cryopreserved as a single cell suspension with almost no loss in post-thaw viability and a slight reduction in plating efficiency when compared with parallel “fresh” cells that were not CryoPaused. The current data indicate that a technical driver enabling this paradigm change is the culture system, wherein an increased recovery with E8 expanded cells using a number of cryopreservation paradigms (Liu and Chen, 2014) was observed.

CryoPause provides a number of advantages compared to conventional PSC culture. CryoPause allows large banks of cells ready for differentiation to be locked in a defined state, enhancing reproducibility. Quality testing, such as measuring contamination (cell lines, mycoplasma, bacteria), genetic instability, synchronization of multiple iPS lines, and purity of culture, can validate entire banks instead of continual surveillance.

Disease modeling studies are best done with multiple iPSC clones derived from numerous healthy and diseased individuals. The conventional parallel culture method is labor intensive and time consuming since maintenance of multiple lines are necessarily done in parallel with directed differentiations to provide a continuous source of fresh starting material for experiments. iPS lines that expand at different rates complicate the synchronous initiation of differentiation and parallel passage, usually resulting in a compromise that maximizes the number of cultures that are ready at one point in time: the remainder are often under or over-expanded. Serial culture also increases the risk of cross-contamination of cell lines, the accidental introduction of microorganisms during experiments, or the use of cells that acquire a genomic abnormality during extended culture. CryoPause separates the work in PSC expansion from the differentiation experiments. It permits repeated differentiations from an identical pool of PSCs, completely eliminating variability in the PSC preparation. A full constellation of quality control criteria such as PSC marker status, genetic integrity, sterility, and cell line authentication can validate each bank before use. Most labs currently perform “spot checks” during use or perhaps before the serial passage even begins. The variable of “just in time” PSC workflows almost certainly reduces the robustness and reproducibility of nearly all PSC applications. It can also be inconvenient since it complicates when a differentiation can be initiated due to uncertainty in the rate of PSC expansion.

The advantages of CryoPause could be even more profound for manufacturing cell therapies. In a typical cell therapy workflow, hPSCs are expanded and banked in a GMP facility before undergoing expensive and time-consuming tests to validate the cell bank. The conversion of this PSC bank into a therapeutically useful cell type usually requires recovery from the cryopreserved state and a limited number of cell passages before initiating differentiation into the therapeutic cell type. This creates the possibility of initiating the differentiation of a cell bank with PSCs in a suboptimal state, potentially reducing the reproducibility and product yield. Manufacturing runs can be exorbitantly expensive in time and money and could potentially cause adverse events in patients. Reproducibility of manufacturing is also one of the key attributes that regulatory authorities examine when assessing a cellular product for human use. Timing, yield and quality of PSC expansion can be completely eliminated as variables for cell therapies if CryoPause can be validated for such applications. It is showed here that CryoPaused WA09 cells can be directed to midbrain dopamine neurons using clinically compatible SOP.

One large complication for using PSC derivatives in some cell replacement therapies is matching the HLA status of cells to patients. Autologous iPSCs are one strategy to create patient-matched cells and avoid immune mismatches. But there are considerable practical and potential safety complications with the autologous strategy. This has led to multiple large-scale efforts to bank large numbers of PSC lines so that they can be carefully quality controlled before clinical use yet still provide a close HLA match to a specific patient for allogeneic transplantation. In this scenario, the use of CryoPausing could simplify the procedures used to convert many different PSCs into clinically useful cells.

### Methods and Materials

### Human Pluripotent Stem Cell Maintenance

WA09 (H9) and 960.1B iPSC lines were initially maintained in Essential 8™ (E8, Thermo Fisher, #A1517001) medium on Geltrex (Thermo Fisher, #A1413202) diluted 1:50 in DMEM/F12 (Thermo Fisher, #11330032), and passaged as clusters every 3-4 days using brief (3 minutes) 0.05% Trypsin-EDTA (Thermo Fisher, #25300054) treatment before scraping (to maintain colony structure) and washing twice with fresh E8 medium. Cells were replated with 10 μM Y-27632 for 24 hours. Cells were used between passage 30-55 and no abnormal karyotypes were found.

FIG. 5D used WA09 cells maintained on MEFs plated at a density of 10,500 cells/cm2, depending on the lot used (Applied StemCell, Inc.). PSCs were fed daily with hPSC media during the week [composed of DMEM/F12, 20% knockout serum replacement (Thermo Fisher, #10828028), 3.5 mM L-glutamine (Thermo Fisher, #25030081), 0.1 mM MEM NEAA (Thermo Fisher, #11140050), 55 μM 2-mercaptoethanol (Thermo Fisher, #21985023), and 6 ng/mL rhFGF basic (R&D Systems, #233-FB)]. On Fridays, cells were often fed with medium supplemented with StemBeads (StemCultures, Inc., SB500), time-released FGF2 that allowed cultures to remain static until a Monday feed. A working protocol for hPSC feeder-based culture is available at http://stemcells.mskcc.org.

### Production of CryoPaused cells

To create CryoPaused cell banks, PSCs grown in E8 medium were dissociated with Accutase (Innovative Cell Technologies, #AT-104) for 30 minutes in a 37° C. incubator. Cells were washed with 2 volumes of E8 (relative to the cells: Accutase) and centrifuged to pellet cells (200×g, room temperature for 5 min). The supernatant was aspirated, and the rinse was repeated. Cells were finally resuspended in FreSR-STM (Stem Cell Technologies, #05859) at 10 million cells/mL unless otherwise indicated. The FreSR-S:cell mixture was added to pre-chilled cryotubes before freezing in a controlled rate freezer using the following program:

Step 1: wait at 4° C.; Step 2: 1.2° C./min (sample) to −4° C.; Step 3: 25° C./min (chamber) to −40° C.; Step 4: 10° C./min (chamber) to −12° C.; Step 5: 1.0° C./min (chamber) to −40° C.; Step 6: 10° C./min (chamber) to −90° C.; Step 7: wait at −90° C. Cryotubes were rapidly transferred to a liquid nitrogen tank once the controlled rate freezer reached −90° C.

The other cryopreservation media used in FIGS. 5B & 5C are PSC Cryopreservation Kit (Thermo Fisher, #A2644601), Stem-CellBanker GMP (AMSBIO, #11890), and 10% DMSO (Sigma Aldrich, #2650) in E8 Medium. Conventional freezing used in FIG. 5C is defined as storing vials in a cell freezing container at −80° C. for 24 hours before transferring to a liquid nitrogen tank.

### Flow Cytometry and Immunofluorescence Analysis for Stem Cell Markers

The Human Pluripotent Stem Cell Sorting and Analysis Kit (BD Biosciences, #560461) and the Human Pluripotent Transcription Factor Analysis Kit (BD Biosciences, #560589) were used as per the manufacturer's protocols to quantify stem cell markers on a BD FACS Aria III. For immunofluorescence staining, the following primary antibodies were used: NANOG (1:200, BD Biosciences, #560482); OCT4 (1:200, Santa Cruz Biotechnology, #sc-9081); SOX2 (1:100, R&D Systems, #AF2018). The appropriate Alexa Fluor-conjugated secondary antibodies were used at 1:400 (Thermo Fisher).

### PluriTest Assay

PluriTest is based on whole genome transcriptome microarray data analysis. Briefly, same procedures as described previously (Müller et al. 2011; Williams et al. 2011; Müller et al. 2012) was employed. RNA was isolated from two biological replicates per culture condition (control and CryoPause, 1×10⁶ cells per sample) using the Qiagen RNeasy isolation kit following the manufacturer's instructions (Qiagen, Hilden, Germany). Illumina HT12v4 microarrays were hybridized following the manufacturer's instructions (Müller et al. 2011; Williams et al. 2011; Müller et al. 2012). The resulting raw data was processed with the R/Bioconductor lumi-package (Du et al. 2008; Lin et al. 2008; Müller et al. 2011). It has been recognized that due to changes in scanner technology and modifications of the hybridization protocol through Illumina, PluriTest results in recent years tend to show lower Pluripotency and higher Novelty Scores (Bernhard Schuldt, University Hospital Schleswig-Holstein, Kiel, Germany, personal communication). Even considering this technical bias, both—control samples and CryoPause samples—pass the empirical Pluripotency and higher Novelty Score thresholds with both biological replicates.

### Reduced Representation Bisulfate Sequencing

For sequencing library preparation, 1 ug of high quality genomic DNA was used with the NEXTFlex Bisulfite-Seq Kit (Bioo, #5119-01), according to the manufacturer instructions. Unmethylated lambda DNA was spiked in at 1% to assess the level of bisulfate conversation. 12 cycles of PCR were performed. Samples were run on Hiseq 2500 Rapid mode Paired End 125, and an average of 140 million reads was generated per sample.

### RRBS DNA Methylation Analysis

FASTQ files were generated by bcl2fastq (V2.17) and filtered for pass filter reads based on Illumina's chastity filter. Sequencing adapters were trimmed by FLEXBAR (V2.4) (Dodt et al. 2012), genomic alignments using Bismark (V0.14.4) (Krueger et al. 2011) and Bowtie2 (V2.2.5) (Langmead et al. 2012) to reference human genome hg19, and per base CpG methylation metrics were calculated with a custom PERL script (Garrett-Bakelmann et al. 2015).

### γH2AX Quantitative Immunofluorescence

Single PSCs were plated in E8 Medium with 10 μM Y-27632 on Geltrex-coated dishes at 100,000 cells/cm2. After 24 hours, cells were treated with 0.5 μM Camptothecin (Sigma, #C9911) in E8 for 1 hour at 37° C. Cells were then stained against phospho-Histone H2A.X (1:300, Millipore, #05-636), and the appropriate Alexa Fluor-conjugated secondary antibodies were used at 1:400. Images were acquired and analyzed on a Perkin-Elmer Operetta using eHarmony software.

### Neural Induction

A derivative of Chambers et al. 2009 was used. Single PSCs were plated in E8 medium with 10 μM Y-27632 on Geltrex-coated dishes (1:30) at 200,000 (fresh control) or 400,000 cells/cm2 (CryoPause) prior to neural induction. After 24 hours, the media was removed and Essential 6™ (E6) Medium (Thermo Fisher, #A1516401) with 100 nM LDN189193 and 10 μM SB431542 (LSB) was added. Cells were fed for three days with E6 Medium+LSB before transitioning to N2 Medium (see below) stepwise over a course of 3 feeds and exchanged with fresh medium every two days. The efficiency of neural conversion was quantitated by flow analysis for OCT4 (BD Biosciences, #560186) and PAX6 (BD Biosciences, #562249). Cultures were also stained using antibodies against OCT4 and PAX6 (1:200, BD Biosciences, #561462). Midbrain dopamine neurons were made using a modification of Kriks et al. 2011. Briefly, WA09 cells grown on Geltrex as above were subjected to dual SMAD inhibition with SHH and WNT signals before eventual withdrawal. After withdrawal, midbrain dopamine supportive medium was added containing the same growth factors and small molecules found in Kriks et al. 2011 (manuscript in preparation). Cultures were stained using antibodies against Tyrosine Hydroxylase (1:1000, Thermo Fisher, #P21962) and FOXA2 (1:200, R&D Systems, #AF2400). The appropriate Alexa Fluor-conjugated secondary antibodies were used at 1:400.

Day 0: E6+100 nM LDN189193 and 10 μM SB431542

Day 1: E6+100 nM LDN189193 and 10 μM SB431542

Day 2: E6+100 nM LDN189193 and 10 μM SB431542

Day 4: 3:1 E6 to N2+100 nM LDN189193 and 10 μM SB431542

Day 6: 1:1 E6 to N2+100 nM LDN189193 and 10 μM SB431542

Day 8: 1:3 E6 to N2+100 nM LDN189193 and 10 μM SB431542

Day 10: N2+100 nM LDN189193 and 10 μM SB431542

N2 media contains 500 mL of DMEM/F12 with 1 g of sodium bicarbonate (Sigma-Aldrich, #S5761), 0.78 g of glucose (Sigma-Aldrich, #G7021), and 0.5 mL of 2-mercaptoethanol (Thermo Fisher, #21985023). The media is sterile filtered (22 μm) before adding 5 mL of N2 Supplement B (Stem Cell Technologies, #07156) and 0.02 nM progesterone (10 μL from 1 mM stock dissolved in 100% ethanol, Sigma-Aldrich, #P8783).

### Mesendoderm Induction

Single PSCs were plated as above. After 24 hours, the media was removed and E6 Medium with 5 μM CHIR99021 (Stemgent #04-0004-10) was added to create mesendoderm (Lam et al. 2014). E6 with 5 μM CHIR99021 was exchanged every 24 hours for up to 4 days. Efficiency of conversion was quantitated by flow analysis for OCT4 (BD Biosciences, #560186) and Brachyury (R&D Systems, #IC2085A). Cultures were stained using antibodies against OCT4 and Brachyury (1:40, R&D Systems, #AF2085), and the appropriate Alexa Fluor-conjugated secondary antibodies were used at 1:400.

### Quantitative RT-PCR

RNA was isolated from cell pellets using an RNase-Free DNase set (Qiagen, #79254) and an RNeasy Mini Kit (Qiagen, #74106). Reverse transcription of RNA samples and cDNA synthesis was performed using an RT2 First Strand Kit (Qiagen, #330404) and the Eppendorf Mastercycler PCR machine. cDNA samples were prepared for qPCR analysis using RT2 SYBR Green Mastermix (Qiagen, #330503). Samples were run on a custom RT2 Profiler PCR Array containing the following Qiagen primers: OTX2 (PPH16151), FOXA2 (PPH00976), CORIN (PPH58029), SHH (PPH02405), LMX1A (PPH22219), LMX1B (PPH12240), EN1 (PPH00986), NR4A2 (PPH02082), NEUROG2 (PPH11564), ASCL1 (PPH07090), TH (PPH02062), CHRNB3 (PPH01891), DDC (PPH19374), CCK (PPH22272), DRD2 (PPH01876), PITX3 (PPH12380), SLC18A (PPH01437), KCNJ6 (PPH01415), SLC17A6 (PPH14888), POU4F1 (PPH14485), NKX6-1 (PPH17340), SIM1 (PPH11011), NKX2-1 (PPH00246), FEV (PPH02033), NKX2-2 (PPH01574), GBX2 (PPH13900), DBH (PPH02066), ISL1 (PPH02461), FOXG1 (PPH01973), HOXB2 (PPH05804), DLX2 (PPH01943), GATA3 (PPH02143), PHOX2A (PPH15630), PHOX2B (PPH10361), PAX6 (PPH02598), FABP7 (PPH02438), MAP2 (PPH02419), POU5F1 (PPH02394), PRR16 (PPH13057), KRT19 (PPH01004), MKI67 (PPH01024), TOP2A (PPH01520), GFAP (PPH02408), ACTB (PPH00073), TBP (PPH01091), HGDC (PPH65835), RTC (PPX63340), and PPC (PPX63339). Analysis was performed using a Bio-Rad c1000 Touch Thermal Cycler CFX96 Real-time System (Bio-Rad, #1855195).

### Teratomas

NSG mice (Jackson Laboratories) were used for in vivo studies and were cared for in accordance with guidelines approved by MSKCC Institutional Animal Care and Use Committee and Research Animal Resource Center. Eight-week-old female mice were injected subcutaneously with 3 million H9 cells in the flank with Matrigel™ (BD Biosciences) mixed 1:2 in HEPES buffered HBSS. Mice were observed daily for signs of morbidity/mortality, and body weights were assessed at least twice weekly. Tumors were measured twice weekly using calipers, and volume was calculated using the formula length×width 2×0.52. At the end of the study, tumors were fixed in 10% formalin, processed, embedded in paraffin, sectioned and stained with hematoxylin and eosin (H&E). Microscopic slides were reviewed by a pathologist.

### Use of Cell Factories

Nunc™ Cell Factory™ System was used, 4 tray layers, (Thermo Fisher, #140004) to expand large banks prior to CryoPausing. Cells were fed with 500 mL of E8 medium for the first two days after passage and 600 mL on the third day. To create a single cell suspension, 140 mL of Accutase was added for 30 minutes at 37° C. then the trays were washed with 100 mL of medium.

### Nucleofections/iCRISPR

To nucleofect CryoPaused cells, the Amaxa™ Cell Line Nucleofector™ Kit V (Lonza, #VCA-1003) was used. WA09 CryoPaused cells were thawed and washed. 100 μL of Solution V was mixed with 22.2 μL of Supplement 1, and 5 million CryoPaused cells were added to 100 μL of this mixture. 10 μl of the GFP control plasmid was added to the reaction before nucleofecting on program B-016 (Lonza Nucleofector™ 2b device). Nucleofected cells were added to E8 with 10 μM Y-27632 on Geltrex as above. Live cells were imaged for FIGS. 3A and 4B and the number of fluorescing cells was determined by treating cultures with Accutase, washing, and resuspending in PBS with 0.1% BSA before measuring fluorescence on a BD FACS Aria III. Flow data was analyzed with FlowJo, and immunofluorescent images were adjusted with Adobe Photoshop.

To perform CryoPaused iCRISPR gene modification, WA01 (H1) iCRISPR cells were expanded as above before 24 hours Dox treatment to induce Cas9 (Gonzalez et al. 2014). Cas9 induced cells were harvested and washed as above before CryoPausing. CryoPaused cells were thawed and washed before nucleofection with 200 ng of HPRT guide RNA purchased from GeneArt (Thermo Fisher). The guide sequence is CATTTCTCAGTCCTAAACA.

### Sendai Transduction

CryoPaused WA09 cells were thawed, washed and resuspended in E8 medium supplemented with 10 μM Y-27632 and Sendai viral vectors expressing EmGFP (CytoTune™ EmGFP, ThermoFisher Scientific, #A16519) were added at a multiplicity of infection of 5. Transduced CP cells were replated and expanded in E8. Transduced cells could be expanded for at least 15 passages.

### REFERENCES

Barker R A, Studer L, Cattaneo E, Takahashi J & G-Force PD consortium. G-Force PD: a global initiative in coordinating stem cell-based dopamine treatments for Parkinson's disease. NPJ Parkinsons Dis. 2015; 1. pii 15017.

Chambers S M, Fasano C A, Papapetrou E P, Tomishima M, Sadelain M, Studer L. Highly efficient neural conversion of human ES and iPS cells by dual inhibition of SMAD signaling. Nat Biotechnol. 2009 March; 27 (3):275-80.

Chen G, Gulbranson D R, Hou Z, Bolin J M, Ruotti V, Probasco M D, Smuga-Otto K, Howden S E, Diol N R, Propson N E, Wagner R, Lee G O, Antosiewicz-Bourget J, Teng J M, Thomson J A. Chemically defined conditions for human iPSC derivation and culture. Nat Methods. 2011 May; 8 (5):424-9.

Dodt M, Roehr J T, Ahmed R, Dieterich C. FLEXBAR-Flexible Barcode and Adapter Processing for Next-Generation Sequencing Platforms. Biology (Basel). 2012; 1 (3):895-905.

Du P, Kibbe W A, Lin S M. lumi: a pipeline for processing Illumina microarray. Bioinformatics. 2008 Jul. 1; 24 (13):1547-8.

Garrett-Bakelman F E, Sheridan C K, Kacmarczyk T J, Ishii J, Betel D, Alonso A, et al. Enhanced reduced representation bisulfite sequencing for assessment of DNA methylation at base pair resolution. J Vis Exp. 2015; (96):e52246.

González F, Zhu Z, Shi Z D, Lelli K, Verma N, Li Q V, Huangfu D. An iCRISPR platform for rapid, multiplexable, and inducible genome editing in human pluripotent stem cells. Cell Stem Cell. 2014 Aug. 7; 15 (2):215-26.

Kacmarczyk T J, Fall M P, Zhang X, Xin Y, Li Y, Alonso A, Betel D. “Same Difference”: Comprehensive evaluation of three DNA methylation measurement platforms. bioRxiv 077941 (2016).

Kriks S, Shim J W, Piao J, Ganat Y M, Wakeman D R, Xie Z, Carrillo-Reid L, Auyeung G, Antonacci C, Buch A, Yang L, Beal M F, Surmeier D J, Kordower J H, Tabar V, Studer L. Dopamine neurons derived from human ES cells efficiently engraft in animal models of Parkinson's disease. Nature. 2011 Nov. 6; 480 (7378):547-51.

Krueger F, Andrews S R. Bismark: a flexible aligner and methylation caller for Bisulfite-Seq applications. Bioinformatics. 2011; 27 (11):1571-2.

Lam A Q, Freedman B S, Morizane R, Lerou P H, Valerius M T, Bonventre J V. Rapid and Efficient Differentiation of Human Pluripotent Stem Cells into Intermediate Mesoderm That Forms Tubules Expressing Kidney Proximal Tubular Markers. J Am Soc Nephrol. 2014 June; 25 (6):1211-25.

Langmead B, Salzberg S. Fast gapped-read alignment with Bowtie 2. Nature Methods. 2012, 9:357-359.

Lin S M, Du P, Huber W, Kibbe W A. Model-based variance-stabilizing transformation for Illumina microarray data. Nucleic Acids Res. 2008 February; 36 (2):e11-1.

Liu W and Chen G. Cryopreservation of human pluripotent stem cells in defined medium. Curr Protoc Stem Cell Biol. 2014 Nov. 3; 31:1C.17.1-1C.17.13.

Müller F-J, Brändi B, Loring J F. Assessment of human pluripotent stem cells with PluriTest. StemBook. Cambridge (Mass.): Harvard Stem Cell Institute; 2012.

Müller F-J, Schuldt B M, Williams R, Mason D, Altun G, Papapetrou E P, et al. A bioinformatic assay for pluripotency in human cells. Nat Methods. 2011 Mar. 6; 8:315-7.

Redon C, Pilch D, Rogakou E, Sedelnikova O, Newrock K, Bonner W. Histone H2A variants H2AX and H2AZ. Curr Opin Genet Dev 2002; 12: 162-169.

Watanabe K, Ueno M, Kamiya D, Nishiyama A, Matsumura M, Wataya T, Takahashi J B, Nishikawa S, Nishikawa S, Muguruma K, Sasai Y. A ROCK inhibitor permits survival of dissociated human embryonic stem cells. Nature Biotechnology. 2007 June; 25 (6):681-6.

Williams R, Schuldt B, Müller F-J. A guide to stem cell identification: Progress and challenges in system-wide predictive testing with complex biomarkers. Bioessays. 2011 Sep. 8.

### Example 2: Cryopreserved Dissociated Pluripotent Stem Cells Express Exogenous Nucleic Acid at a Greater Level Than Non-cryopreserved Cells

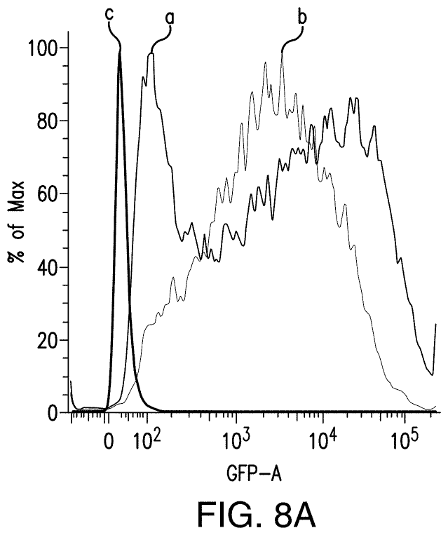

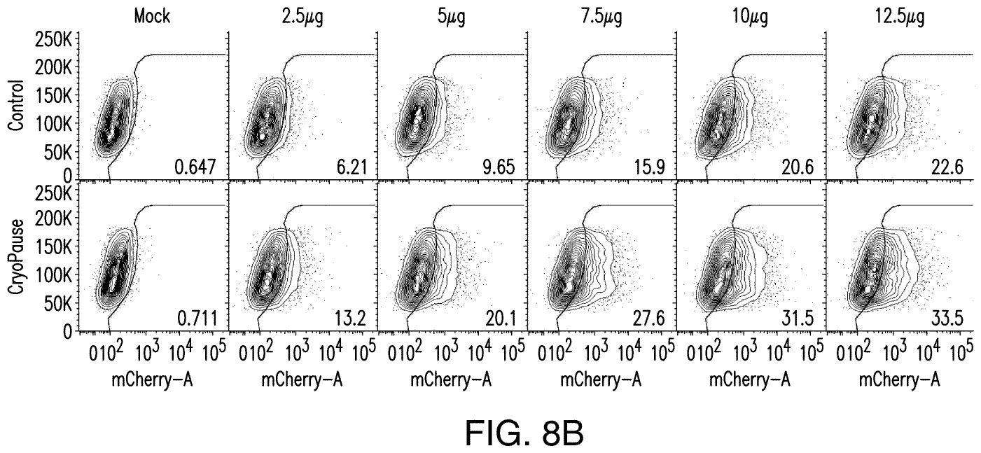

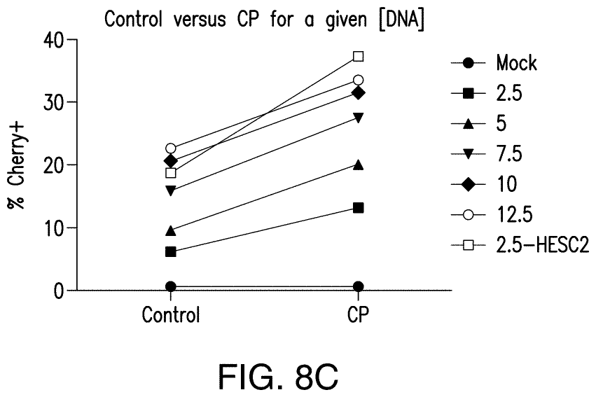

WA09 human embryonic stem cells were cryopreserved, thawed and immediately nucleofected post-thaw with a 3.4 kb GFP plasmid, as described by Example 1. Fresh non-frozen control WA09 cells that had not been cryopreserved were similarly nucleofected, and GFP expression was analyzed 24 hours post-nucleofection using flow cytometry. Non-transfected WA09 cells were also used as a negative control. As shown by FIG. 8A, the CryoPaused cells exhibited a different pattern of small DNA uptake: overall, slightly fewer cells expressed GFP from the small plasmid, but those that did were brighter, suggesting that CryoPause makes cells more competent to take up large amounts of DNA. The lower percentage with the smaller construct in the entire pool of cells might be a result of titration of DNA: that is, cells that take up large amounts of plasmid might leave little for the cells in the population that would normally take up less DNA

WA09 CryoPaused and fresh non-frozen cells were also nucleofected with a larger 9.3 kb CRISPR/Cas9 plasmid that expresses mCherry. Cells were nucleofected with 2.5 μg, 5 μg, 7.5 μug, 10 μg and 12.5 μg of plasmid. A second nucleofection solution, HSC2, was also tested with 2.5 μg of the plasmid under CryoPause and fresh non-frozen conditions. As shown by FIGS. 8B and 8C, the HSC-buffer CryoPaused WA09 cells exhibited a higher level of expression at all concentrations compared to the fresh non-frozen cells.

Although the presently disclosed subject matter and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, and composition of matter, means, methods and steps described in the specification. As one of ordinary skill in the art will readily appreciate from the disclosure of the presently disclosed subject matter, processes, machines, manufacture, compositions of matter, means, methods, or steps, presently existing or later to be developed that perform substantially the same function or achieve substantially the same result as the corresponding embodiments described herein may be utilized according to the presently disclosed subject matter. Accordingly, the appended claims are intended to include within their scope such processes, machines, manufacture, compositions of matter, means, methods, or steps.

Patents, patent applications, publications, product descriptions and protocols are cited throughout this application the disclosures of which are incorporated herein by reference in their entireties for all purposes.

What is claimed is:

1. A composition comprising a frozen population of dissociated cells and a cryopreservation medium, wherein the concentration of cells in the frozen population is at least about 1 million cells/ml.

2. A composition comprising a cell transfected with a heterologous nucleic acid, prepared by transfecting a cell obtained by thawing a frozen population of dissociated cells and a cryopreservation medium, wherein the concentration of cells in the frozen population is at least about 1 million cells/ml.

3. The composition of claim 1, wherein the concentration of cells in the frozen population is at least about 5 million cells/ml, at least about 30 million cells/ml, or at least about 50 million cells/ml.

4. The composition of claim 1, wherein the cells are mammalian cells.

5. The composition of claim 4, wherein the mammalian cells are pluripotent stem cells (PSCs).

6. The composition of claim 5, wherein the pluripotent stem cells are induced pluripotent stem cells (iPSCs) or embryonic stem cells (ESCs).

7. The composition of claim 1, wherein the population of dissociated cells expresses an exogenous nucleic acid, wherein the level of expression of the exogenous nucleic acid is greater than the expression level of the exogenous nucleic acid by a population of cells that has not been frozen.

8. The composition of claim 7, wherein the level of expression of the population of dissociated cells is at least about 2 times greater or at least about 5% greater than the level of expression of the population of cells that has not been frozen.

9. A method of preparing a composition comprising frozen cells, comprising:
dissociating a population of cells cultured in a culture medium;
suspending the dissociated cells in a cryopreservation medium to form a cell suspension; and
freezing the cell suspension to form a composition of frozen cells,
wherein the composition of frozen cells has a concentration of at least about 1 million cells/ml.

10. A method of preparing a transfected cell, comprising
(i) preparing a composition comprising frozen cells by a method comprising:
dissociating a population of cells cultured in a culture medium;
suspending the dissociated cells in a cryopreservation medium to form a cell suspension; and
freezing the cell suspension to form a composition of frozen cells,
wherein the composition of frozen cells has a concentration of at least about 1 million cells/ml; and
(ii) transfecting a cell from composition (i) with a heterologous nucleic acid.

11. The method of claim 9, wherein the step of dissociating a population of cells further comprises exposing the population of cells to an effective amount of a cell dissociation solution.

12. The method of claim 11, wherein the cell dissociation solution is selected from the group consisting of an enzyme-free cell dissociation solution and an enzyme-containing solution.

13. The method of claim 12, wherein the enzyme-containing cell dissociation solution comprises one or more enzymes.

14. The method of claim 12, wherein the enzyme-free cell dissociation solution comprises one or more chelating agent.

15. The method of claim 9, further comprising introducing a nucleic acid into the population of cells that are subject to frozen.

16. An in vitro method of culturing cells, comprising:
thawing a composition of frozen cells comprising a population of dissociatd cells and a cryopreservation medium; and
subjecting the cells to a downstream treatment, wherein the cells are not subjected to exponential expansion before the downstream treatment.

17. The method of claim 16, wherein the composition of frozen cells has a concentration of at least about 1 million cells/ml, at least about 5 million cells/ml, or at least about 30 million cells/ml.

18. The method of claim 16, wherein the downstream treatment comprises an in vitro method of differentiating the cells.

19. The method of claim 18, wherein the cells are differentiated into a plurality of dopamine-producing precursor cells.

20. The method of claim 19, wherein the dopamine-producing precursor cells express detectable levels of forkhead box protein A2 (FOXA2), LIM homeobox transcription factor 1 alpha (LMX1A), tyrosine hydroxylast (TH), or combinations thereof.
